(19) United States
(12) Patent Application Publication
(10) Pub. No.: US 2022/0287553 A1
(43) Pub. Date: Sep. 15, 2022

(54) SYSTEMS AND METHODS FOR MEDICAL IMAGING

(71) Applicant: ACTIV SURGICAL, INC., Boston, MA (US)

(72) Inventors: Stephen TULLY, Boston, MA (US); John OBERLIN, Boston, MA (US); Emanuel DEMAIO, Boston, MA (US); Liam O'SHEA, Boston, MA (US); Thomas CALEF, Boston, MA (US)

(21) Appl. No.: 17/752,617

(22) Filed: May 24, 2022

Related U.S. Application Data
(63) Continuation of application No. PCT/US20/62086, filed on Nov. 24, 2020.
(60) Provisional application No. 62/939,969, filed on Nov. 25, 2019.

(51) Int. Cl. A61B 1/06

(52) U.S. Cl. CPC A61B 1/0655; A61B 1/0684; A61B 1/063; A61B 1/0638; A61B 1/0653

(57) ABSTRACT

The present disclosure provides systems and methods for medical imaging. The system may comprise a plurality of illumination sources. The plurality of illumination sources may comprise at least two of (i) a white light source configured to generate a white light beam and (ii) one or more light emitting diodes (LEDs) or laser light sources configured to generate one or more laser light beams. The system may further comprise a movable plate comprising one or more cut-outs. The movable plate may be (i) optically aligned with one or more of the plurality of illumination sources and (ii) configured to move so as to (a) control an exposure of the one or more illumination sources through the one or more cut-outs, relative to a pre-determined frame capture rate, and (b) generate one or more light pulses based on the controlled exposure of the one or more illumination sources.

### CROSS-REFERENCE

This application is a continuation application of International Patent Application PCT/US2020/062086, filed on Nov. 24, 2020, which claims priority to U.S. Provisional Patent Application No. 62/939,969, filed on Nov. 25, 2019, each of which is incorporated herein by reference in its entirety for all purposes.

### BACKGROUND

Medical imaging technology may be used to capture images or video data of internal anatomical features of a subject or patient during medical or surgical procedures. The images or video data captured may be processed and manipulated to provide surgeons and medical operators with a visualization of internal structures or processes within a patient or subject. Conventional medical imaging systems available today may use one or more dyes to help visualize internal processes such as blood flow. Such systems may limit the time frame during which an operator may visualize changes in blood flow.

### SUMMARY

Recognized herein are various limitations with medical imaging systems currently available. The present disclosure provides systems and methods to address existing shortcoming or deficiencies of conventional medical imaging systems. The systems and methods disclosed herein may be used to enhance medical imaging by selectively controlling the exposure of multiple illumination sources through one or more cut-outs of a movable plate and combining pulses of light from different multi-spectral illumination sources. As such, the system and methods disclosed herein may be implemented to visualize and digitally map anatomical structures within a patient in three-dimensional (3D) perspective, in real-time and without the use of dyes, thereby providing medical operators with additional visual information (e.g., a real-time visual depiction of a patient's blood perfusion) that can inform or guide them during a surgical procedure.

In an aspect, the present disclosure provides a system for illuminating a target region in a subject's body. The system may comprise: a plurality of illumination sources comprising at least two of (i) a white light source configured to generate a white light beam and (ii) one or more light emitting diodes (LEDs) or laser light sources configured to generate one or more laser light beams; and a movable plate comprising one or more cut-outs, wherein the movable plate is (i) optically aligned with one or more of the plurality of illumination sources and (ii) configured to move so as to (a) control an exposure of the one or more illumination sources through the one or more cut-outs, relative to a pre-determined frame capture rate, and (b) generate one or more light pulses based on the controlled exposure of the one or more illumination sources.

In some embodiments, the movable plate may be configured to rotate relative to the one or more illumination sources along an optical axis. In some embodiments, the movable plate may comprise a low transmittance material that is configured to prevent transmission of light through one or more solid portions of the movable plate.

In some embodiments, the one or more cut-outs may comprise a notch on the movable plate. In some embodiments, the one or more cut-outs may comprise a plurality of notches arranged on different portions of the movable plate. In some embodiments, the one or more cut-outs may comprise one or more annular-shaped openings on the movable plate.

In some embodiments, the one or more laser light sources may comprise two or more laser light sources that are configured to generate two or more laser light beams having different wavelengths. In some embodiments, the two or more laser light sources may comprise a gas laser, a chemical laser, a liquid laser, a dye laser, a metal-vapor laser, a solid-state laser, or a semiconductor laser. In some embodiments, the two or more laser light sources may comprise an infrared laser, a near-infrared laser, a short-wavelength infrared laser, a mid-wavelength infrared laser, a long-wavelength infrared laser, or a far-infrared laser. In some embodiments, the two or more laser light sources may be configured to generate two or more laser light beams with a wavelength between about 700 nanometers (nm) and about 1 millimeter (mm).

In some embodiments, the movable plate may be optically aligned with the one or more laser light sources. In some embodiments, the movable plate and the white light source may not share a common optical axis. In some embodiments, the white light source may be disposed relative to the movable plate such that the white light beam does not pass through the movable plate. In some embodiments, the white light beam from the white light source may be transmitted continuously without being affected or separated into pulses by the movable plate. In some embodiments, the one or more light pulses may be obtained from the one or more laser light beams.

In some embodiments, the system may further comprise a light aggregation module configured to (i) combine (a) the one or more light pulses obtained from the one or more laser light beams with (b) the white light beam to generate (c) a combined light beam, and (ii) provide the combined light beam to a scope. The scope may be insertable into the subject's body and configured to direct the combined light beam onto the target region.

In some embodiments, the movable plate may be optically aligned with (i) the white light source and (ii) the one or more laser light sources. In some embodiments, the one or more light pulses may be obtained from (i) the white light beam and (ii) the one or more laser light beams.

In some embodiments, the system may further comprise a light aggregation module configured to (i) combine (a) the one or more light pulses obtained from the white light beam with (b) the one or more light pulses obtained from the one or more laser light beams, to generate (c) a combined light beam, and (ii) provide the combined light beam to a scope. The scope may be insertable into the subject's body and configured to direct the combined light beam onto the target region.

In some embodiments, the plurality of illumination sources may further comprise (iii) an indocyanine green (ICG) excitation light source configured to generate an ICG excitation light beam. In some embodiments, the ICG excitation light source may be disposed relative to the movable plate such that the ICG excitation beam does not pass through the movable plate. In some embodiments, the movable plate may be optically aligned with (i) the one or more laser light sources. In some embodiments, the movable plate and the white light source may not share a common optical axis. In some embodiments, the white light beam from the white light source may be transmitted continuously without being affected or separated into pulses by the movable plate. In some embodiments, the movable plate and the ICG excitation light source may not share a common optical axis. In some embodiments, the ICG excitation light beam from the ICG excitation light source may be transmitted continuously without being affected or separated into pulses by the movable plate. In some embodiments, the one or more light pulses may be obtained from (i) the one or more laser light beams.

In some embodiments, the system may further comprise a light aggregation module configured to (i) combine (a) the one or more light pulses obtained from the one or more laser light beams with (b) at least one of the white light beam or the ICG excitation light beam to generate (c) a combined light beam, and (ii) provide the combined light beam to a scope. The scope may be insertable into the subject's body and configured to direct the combined light beam onto the target region.

In some embodiments, the movable plate may be optically aligned with (i) the one or more laser light sources and (ii) the ICG excitation light source. In some embodiments, the movable plate and the white light source may not share a common optical axis. In some embodiments, the white light beam from the white light source may be transmitted continuously without being affected or separated into pulses by the movable plate. In some embodiments, the one or more light pulses may be obtained from (i) the one or more laser light beams and (ii) the ICG excitation light beam.

In some embodiments, the system may further comprise a light aggregation module configured to (i) combine (a) the one or more light pulses obtained from the one or more laser light beams and the ICG excitation light beam with (b) the white light beam to generate (c) a combined light beam, and (ii) provide the combined light beam to a scope. The scope may be insertable into the subject's body and configured to direct the combined light beam onto the target region.

In some embodiments, the movable plate may be optically aligned with (i) the one or more laser light sources and (ii) the white light source. In some embodiments, the movable plate and the ICG excitation light source may not share a common optical axis. In some embodiments, the ICG excitation light beam from the ICG excitation light source may be transmitted continuously without being affected or separated into pulses by the movable plate. In some embodiments, the one or more light pulses may be obtained from (i) the one or more laser light beams and (ii) the white light beam.

In some embodiments, the system may further comprise a light aggregation module configured to (i) combine (a) the one or more light pulses obtained from the one or more laser light beams and the white light beam with (b) the ICG excitation light beam to generate (c) a combined light beam, and (ii) provide the combined light beam to a scope. The scope may be insertable into the subject's body and configured to direct the combined light beam onto the target region.

In some embodiments, the movable plate may be optically aligned with (i) the one or more laser light sources, (ii) the white light source, and (iii) the ICG excitation light source. In some embodiments, the one or more light pulses may be obtained from (i) the one or more laser light beams, (ii) the white light beam, and (iii) the ICG excitation light beam.

In some embodiments, the system may further comprise a light aggregation module configured to (i) combine (a) the one or more light pulses obtained from the one or more laser light beams with (b) the one or more light pulses obtained from the white light beam and the ICG excitation light beam to generate (c) a combined light beam, and (ii) provide the combined light beam to a scope. The scope may be insertable into the subject's body and configured to direct the combined light beam onto the target region.

In some embodiments, the one or more cut-outs may correspond to one or more open regions disposed on the movable plate. In some embodiments, the one or more open regions may be configured to allow a transmission of light through the movable plate when the one or more cut-outs are aligned with at least one of said plurality of illumination sources.

In some embodiments, the movable plate may be configured to control the exposure of the one or more illumination sources by selectively allowing one or more light beams generated by the one or more illumination sources to pass through the one or more cut-outs of the moveable plate during one or more pre-determined time intervals.

In some embodiments, the one or more light beams may comprise the one or more laser light beams, the white light beam, or the ICG excitation light beam.

In some embodiments, the one or more pre-determined time intervals may be determined based on (i) a rotation speed of the movable plate and (ii) a cut-out geometry associated with the one or more cut-outs.

In some embodiments, at least one of the plurality of illumination sources may be aligned with at least one of the one or more cut-outs during the pre-determined time intervals.

In some embodiments, the one or more open regions may comprise one or more distinct open regions configured to provide one or more distinct exposure times for at least one of the plurality of illumination sources while the movable plate rotates relative to the plurality of illumination sources.

In some embodiments, the one or more open regions may comprise one or more annular-shaped openings disposed at one or more radial distances from a center of the movable plate. In some embodiments, each of the one or more radial distances may correspond respectively to at least one of the plurality of illumination sources.

In some embodiments, the one or more annular-shaped openings may be disposed at one or more distinct angular positions relative to each other. In some embodiments, a first annular-shaped opening of the one or more annular-shaped openings may have a first circumferential length that is different than a second circumferential length of a second annular-shaped opening of the one or more annular-shaped openings.

In some embodiments, the one or more open regions may comprise one or more wedge-shaped opening with a circumferential width that is configured to provide a pre-determined exposure time for each of said plurality of illumination sources. In some embodiments, the one or more wedge-shaped openings may be disposed at one or more distinct angular positions relative to each other. In some embodiments, a first wedge-shaped opening of the one or more wedge-shaped openings may have a first circumferential width that is different than a second circumferential width of a second wedge-shaped opening of the one or more wedge-shaped openings. In some embodiments, the one or more distinct open regions may comprise (i) a first open region configured to expose at least one of the plurality of illumination sources for a first pre-determined time interval, and (ii) a second open region configured to expose at least one of the plurality of illumination sources for a second pre-determined time interval. In some embodiments, the first open region may have a different geometry than the second open region.

In some embodiments, the movable plate may be configured to rotate at a pre-determined rate of rotation such that at least a subset of the plurality of illumination sources is exposed for one or more time intervals corresponding to an imaging period during which an imaging device with the pre-determined frame capture rate is configured to acquire one or more image frames.

In some embodiments, the imaging device may comprise an image sensor or a camera.

In some embodiments, the system may further comprise an additional movable plate configured to rotate relative to the plurality of illumination sources and the movable plate. In some embodiments, the additional movable plate may be configured to rotate at a second rate that is different than a first rate at which the movable plate is configured to rotate. In some embodiments, the additional movable plate may be configured to rotate in a second direction that is different than a first direction in which the movable plate is configured to rotate. In some embodiments, the movable plate may comprise a first set of cut-outs with a different geometry or arrangement than a second set of cut-outs on the additional movable plate.

In some embodiments, the scope may comprise a laparoscope, an endoscope, a borescope, a videoscope, or a fiberscope.

In some embodiments, the one or more light pulses may be provided to the light aggregation module via one or more optical fiber bundles.

In some embodiments, the white light source may be provided in a separate illumination module that is located remote from one or more illumination sources of the plurality of illumination sources.

In some embodiments, the synchronization of the exposure of the one or more illumination sources through the one or more cut-outs relative to the pre-determined frame capture rate may be performed using a timing signal generated using one or more photointerrupters. In some embodiments, the synchronization of the exposure of the one or more illumination sources through the one or more cut-outs relative to the pre-determined frame capture rate may be performed using a timing signal generated by an imaging device.

In another aspect, the present disclosure provides a method for illuminating a target region of a subject. The method may comprise: providing a plurality of illumination sources comprising (i) a white light source configured to generate a white light beam and (ii) one or more laser light sources configured to generate one or more laser light beams; directing one or more light beams generated by the plurality of illumination sources towards a movable plate comprising one or more cut-outs, wherein the movable plate is (i) optically aligned with one or more of the plurality of illumination sources, and (ii) configured to move so as to (a) control an exposure of the one or more illumination sources through the one or more cut-outs, relative to a pre-determined frame capture rate, and (b) generate one or more light pulses based on the controlled exposure of the one or more illumination sources; and providing the one or more light pulses to a light aggregation module, wherein the light aggregation module is configured to (i) combine the one or more light pulses obtained from each of the one or more light beams generated by the plurality of illumination sources to generate a combined light beam, and (ii) provide the combined light beam to a scope, wherein the scope is insertable into the subject's body and configured to direct the combined light beam onto the target region.

In some embodiments, the plurality of illumination sources may further comprise an indocyanine green (ICG) excitation light source configured to generate an ICG excitation light beam.

In another different aspect, the present disclosure provides a system for illuminating a target region of a subject's body. The system may comprise: a plurality of illumination sources comprising at least two of (i) a white light source configured to generate a white light beam and (ii) one or more light emitting diodes (LEDs) or laser light sources configured to generate one or more laser light beams; and a movable plate comprising one or more cut-outs, wherein the movable plate is optically aligned with one or more of the plurality of illumination sources and configured to (i) move relative to the one or more illumination sources and (ii) control a pulsing of the one or more illumination sources in synchronization with a pre-determined frame capture rate.

In some embodiments, the movable plate may be configured to control the pulsing of the one or more illumination sources by adjusting one or more time intervals during which each of the plurality of illumination sources is optically aligned with the one or more cut-outs of the movable plate.

Additional aspects and advantages of the present disclosure will become readily apparent to those skilled in the art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIGS. 1A and 1B schematically illustrate a plurality of illumination sources, in accordance with some embodiments.

FIGS. 2A and 2B schematically illustrate a movable plate, in accordance with some embodiments.

FIGS. 2C, 2D, and 2E schematically illustrate a movable plate and an additional movable plate, in accordance with some embodiments.

FIG. 3A schematically illustrates a system for illuminating a target region in a subject's body, in accordance with some embodiments.

FIG. 3B schematically illustrates a fiber bundle for aggregating light pulses, in accordance with some embodiments.

FIG. 4 schematically illustrates a medical imaging system configured to generate an image of a target region illuminated by the plurality of illumination sources, in accordance with some embodiments.

FIGS. 5A and 5B schematically illustrate a movable plate configured to control an exposure of one or more laser light sources, in accordance with some embodiments.

FIGS. 6A and 6B schematically illustrate an exposure of one or more laser light sources that are optically aligned with the movable plate, in accordance with some embodiments.

FIGS. 7A and 7B schematically illustrate an indocyanine green (ICG) excitation light source that is not optically aligned with a movable plate, in accordance with some embodiments.

FIG. 8 schematically illustrates an exposure of one or more laser light sources and an ICG excitation light source that is not optically aligned with the movable plate, in accordance with some embodiments.

FIGS. 9A and 9B schematically illustrate a movable plate configured to control an exposure of an ICG excitation light source and one or more laser light sources, in accordance with some embodiments.

FIG. 10 schematically illustrates an exposure of one or more laser light sources and an ICG excitation light source that is optically aligned with the movable plate, in accordance with some embodiments.

FIGS. 11A and 11B schematically illustrate a movable plate configured to control an exposure of a white light source and one or more laser light sources, in accordance with some embodiments.

FIG. 12 schematically illustrates a light aggregation module configured to combine one or more light beams generated by a white light source and a laser light source, in accordance with some embodiments.

FIG. 13 schematically illustrates a method for illuminating a target region in a subject's body, in accordance with some embodiments.

FIG. 14 schematically illustrates a computer system that is programmed or otherwise configured to implement methods provided herein.

### DETAILED DESCRIPTION

While various embodiments of the present disclosure have been shown and described herein, it will be obvious to those skilled in the art that such embodiments are provided by way of example only. Numerous variations, changes, and substitutions may occur to those skilled in the art without departing from the embodiments of the present disclosure. It should be understood that various alternatives to the embodiments of the present disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “perfusion,” as used herein, generally refers to passage of fluid through the circulatory system or lymphatic system to an organ or a tissue. In an example, perfusion may refer to the delivery of blood at the level of the arteries or capillaries, in which exchange of oxygen and/or nutrients between blood and tissue takes place. In some cases, perfusion may comprise flow rate of the fluid, volume of the fluid that is present or traversing across a target tissue site, a pattern of flow channels of the fluid at the target tissue site, or a combination thereof. In some cases, perfusion of the liquid of interest may be increasing, decreasing, or remaining substantially the same during one or more imaging processes. In some cases, any change in flow rate or volume of the perfusing fluid may be indicative of (i) one or more biological events or (ii) one or more surgical events occurring upstream of, downstream of, or substantially at the target tissue site. When quantified, perfusion may be measured as the rate at which blood is delivered to tissue, or volume of blood per unit time (blood flow) per unit tissue mass, in units of cubic meter per second per kilogram (m3/s/kg) or milliliters per minute per grams (mL/min/g). Degree of perfusion may be indicative of one or more health conditions, e.g., cardiovascular disease such as coronary artery disease, cerebrovascular disease, peripheral artery disease, etc.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 0.0001 millisecond (ms), 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

The present disclosure provides systems and methods for enhancing medical imaging technology. Conventional medical imaging systems available today may use one or more dyes to help visualize internal processes such as blood flow, but such systems may limit the time frame during which an operator may visualize changes in a patient's blood flow. The systems and methods disclosed herein may be used to enhance medical imaging by selectively controlling the exposure of multiple illumination sources through one or more cut-outs on a movable plate and combining pulses of light from different multi-spectral illumination sources. As such, the system and methods disclosed herein may be implemented to visualize and digitally map anatomical structures within a patient in three-dimensional (3D) perspective, in real-time and without the use of dyes, thereby providing medical operators with additional visual information (e.g., a real-time visual depiction of a patient's blood perfusion) that can inform or guide them during a surgical procedure.

In an aspect, the present disclosure provides a system for illuminating a target region in a subject's body. The system may comprise a plurality of illumination sources comprising (i) a white light source configured to generate a white light beam and (ii) one or more laser light sources configured to generate one or more laser light beams; and a movable plate comprising one or more cut-outs, wherein the movable plate is (i) optically aligned with one or more of the plurality of illumination sources and (ii) configured to move so as to (a) control an exposure of the one or more illumination sources through the one or more cut-outs, relative to a pre-determined frame capture rate, and (b) generate one or more light pulses based on the controlled exposure of the one or more illumination sources.

As used throughout this specification, controlling an exposure of multiple illumination sources may refer to controlling a pulsing of the multiple illumination sources using a movable plate comprising one or more cut-outs. As such, controlling the exposure of multiple illumination sources may be referred to interchangeably as controlling the pulsing of multiple illumination sources. Controlling the pulsing of multiple illumination sources may comprise adjusting one or more time intervals during which each of a plurality of illumination sources is optically aligned with one or more cut-outs of the movable plate.

The target region may be a region within a subject (e.g., a human, a child, an adult, a medical patient, a surgical patient, etc.) that may be illuminated by one or more illumination sources. The target region may be a region within the subject's body. In some cases, the target region may correspond to an organ of the subject, a vasculature of the subject, or any anatomical structure of the subject. In some cases, the target region may correspond to a portion of an organ, a vasculature, or an anatomical structure of the subject.

In some cases, the target region may be a region on a portion of the subject's body. The region may comprise a portion of an epidermis, a dermis, and/or a hypodermis of the subject. In other cases, the target region may correspond to a wound located on the subject's body. The wound may be a burn wound. Alternatively, the target region may correspond to an amputation site of the subject. In any of the embodiments described herein, the target region may correspond to a portion of a subject's body that receives blood flow. The target region may be an organ inside a subject's body or an anatomical feature of a subject's body.

The systems and methods of the present disclosure may allow visualization of structures or features (e.g., blood flow) that are in a target region, near a target region, and/or beneath a surface of a target region, which structures or features would ordinarily be invisible to the human eye or other scope assemblies. The systems and methods of the present disclosure may allow visualization of one or more anatomical structures and/or physiological features or functions. The systems and methods of the present disclosure may be used for physiologic, pathologic, morphologic, and/or anatomic visualizations of various structures, features, and/or functions within a subject's body. The systems and methods of the present disclosure may be used to visualize one or more invisible features in a target region. The systems and methods of the present disclosure may enable a plurality of different imaging modalities. For example, the systems and methods of the present disclosure may enable laser speckle imaging capabilities as well as dye-based imaging and/or white-light based imaging (i.e., RGB photographic images and/or videos). In some cases, the systems and methods of the present disclosure may allow users to switch between different visualization modes, e.g., (i) white-light based video only, (ii) laser speckle imaging only, (iii) dye-based imaging, (iv) both white-light based imaging and laser speckle imaging, or (v) any combination thereof.

The plurality of illumination sources may comprise a white light source. The white light source may comprise a lamp (e.g., an incandescent lamp, a fluorescent lamp, a compact fluorescent lamp, a halogen lamp, a metal halide lamp, a fluorescent tube, a neon lamp, a high intensity discharge lamp, or a low pressure sodium lamp), a light bulb (e.g., an incandescent light bulb, a fluorescent light bulb, a compact fluorescent light bulb, or a halogen light bulb), and/or a light emitting diode (LED). The white light source may be configured to generate a white light beam. The white light beam may be a polychromatic emission of light comprising one or more wavelengths of light. The one or more wavelengths of light may correspond to a visible spectrum of light. The one or more wavelengths of light may have a wavelength between about 400 nanometers (nm) and about 700 nanometers (nm). In some cases, the white light beam may be used to generate an RGB image of a target region of a subject.

The plurality of illumination sources may comprise one or more laser light sources. In some cases, the one or more laser light sources may comprise an infrared (IR) laser, a near-infrared laser, a short-wavelength infrared laser, a mid-wavelength infrared laser, a long-wavelength infrared laser, and/or a far-infrared laser.

As described elsewhere herein, the one or more laser light sources may be configured to generate one or more laser light beams. In such cases, the one or more laser light sources may be configured to operate as a continuous wave laser. A continuous wave (CW) laser may be a laser that is configured to produce a continuous, uninterrupted beam of light with a stable output power. Continuous-wave (CW) operation of a laser means that the laser is continuously pumped and may continuously emits pulses of light and/or energy. The emission can occur in a single resonator mode (i.e., a single-frequency operation) or on multiple resonator modes. In some cases, the laser may be configured to operate as a quasi-continuous-wave laser, in which case the laser may only be switched on for limited time intervals.

The one or more light emitting diodes (LEDs) or laser light sources may be configured to generate one or more laser light beams with a wavelength between about 700 nanometers (nm) and about 1 millimeter (mm). In some cases, the one or more laser light beams may be generated using one or more visible light laser diodes and/or one or more infrared laser diodes. In such cases, the one or more laser light beams may have a wavelength between about 350 nanometers and about 2.5 micrometers (μm). For example, the one or more laser light beams may have a wavelength of at least about 350 nm, 400 nm, 450 nm, 500 nm, 550 nm, 600 nm, 650 nm, 700 nm, 750 nm, 800 nm, 850 nm, 900 nm, 950 nm, 1000 nm, 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm, 2.3 μm, 2.4 μm, 2.5 μm, or more.

In some cases, the one or more laser light sources may comprise two or more laser light sources that are configured to generate two or more laser light beams having different wavelengths. The two or more laser light beams may have a wavelength between about 700 nanometers (nm) and about 1 millimeter (mm).

As described above, the plurality of illumination sources may comprise one or more laser light sources. In some cases, the one or more laser light sources may comprise a solid-state laser, a gas laser, a liquid laser, and/or a semiconductor laser.

In some cases, the one or more laser light sources may comprise a solid-state laser. A solid-state laser may be a laser that uses a solid material (e.g., glass or a crystalline material) as a laser medium. The solid-state laser may be a ruby laser, a Nd:YAG laser, a NdCrYAG laser, an Er:YAG laser, a neodymium YLF (Nd:YLF) solid-state laser, a neodymium doped Yttrium orthovanadate (Nd:YVO4) laser, a neodymium doped yttrium calcium oxoborate Nd:YCa4O(BO3)3 (Nd:YCOB) laser, a neodymium glass (Nd:Glass) laser, a titanium sapphire (Ti:sapphire) laser, a thulium YAG (Tm:YAG) laser, a ytterbium YAG (Yb:YAG) laser, a ytterbium:203 (glass or ceramics) laser, a ytterbium doped glass laser (rod, plate/chip, and fiber), a holmium YAG (Ho:YAG) laser, a chromium ZnSe (Cr:ZnSe) laser, a cerium doped lithium strontium (or calcium) aluminum fluoride (Ce:LiSAF, Ce:LiCAF) laser, a promethium 147 doped phosphate glass solid-state laser, a chromium doped chrysoberyl(alexandrite) laser, an erbium doped laser, an erbium—ytterbiumcodoped glass lasers, a trivalent uranium doped calcium fluoride (U:CaF2) solid-state laser, a divalent samarium doped calcium fluoride (Sm:CaF2) laser, and/or an F-Center laser.

In some cases, the one or more laser light sources may comprise a gas laser. A gas laser may be a laser in which an electric current is discharged through a gas inside a laser medium to produce laser light. The gas laser may be an argon laser, a carbon dioxide laser, a carbon monoxide laser, an excimer laser, a helium laser, a helium-neon laser, a krypton laser, a nitrogen laser, or a xenon laser.

In some cases, the one or more laser light sources may comprise a liquid laser. A liquid laser may be a laser that uses a liquid as a laser medium.

In some cases, the one or more laser light sources may comprise a dye laser. A dye laser may use different organic dyes to produce emissions from the ultraviolet to near infrared spectrum. A dye laser may be operated in the visible with tunable emissions of red, yellow, green, or blue laser emission at almost any wavelength. The dye laser may use Rhodamine-6G in solution.

In some cases, the one or more laser light sources may comprise a semiconductor laser. A semiconductor laser may be a laser that uses a p-n junction of a semiconductor diode as the laser medium. The semiconductor laser may be a semiconductor laser diode, a GaN laser, an InGaN laser, an AlGaInP, an AlGaAs, an InGaAsP, a lead salt laser, a vertical cavity surface emitting laser (VCSEL), a quantum cascade laser, and/or a hybrid silicon laser.

In some cases, the one or more laser light sources may comprise a chemical laser. A chemical laser may include a hydrogen fluoride laser, a deuterium flouride laser, a chemical oxygen-iodine laser, or an all gas-phase iodine laser. In other cases, the laser may be a metal-vapor laser. The metal-vapor laser may be a helium—cadmium (HeCd) metal-vapor laser, a helium—mercury (HeHg) metal-vapor laser, a helium—selenium (HeSe) metal-vapor laser, a helium—silver (HeAg) metal-vapor laser, a strontium Vapor laser, a neon—copper (NeCu) metal-vapor laser, a copper vapor laser, a gold vapor laser, and/or a manganese (Mn/MnCl2) vapor laser. Alternatively, the laser may be a free electron laser, a gas dynamic laser, a Samarium laser, a Raman laser, and/or a nuclear pumped laser.

In some cases, the one or more laser light sources may comprise an excimer laser. An excimer laser may use reactive gases such as chlorine and fluorine mixed with inert gases such as argon, krypton, or xenon. When electrically stimulated, the reactive gases may produce a pseudomolecule or dimer. When exposed to the laser, the dimer may produce light in the ultraviolet range of the electromagnetic spectrum.

In some cases, one or more laser light sources may be used to illuminate a target region located inside a subject's body. Optionally, in other cases, one or more laser light sources may be used to perform one or more surgical procedures (e.g., heating of tissue, removal of tissue, and/or ablation of tissue). The one or more laser light sources used to illuminate a target region in a subject's body may or may not be different than the one or more laser light sources used to perform one or more surgical procedures on the subject or patient. In some cases, the one or more laser light sources may be configured to operate in a first state or a second state. The first state may configure the laser light source to illuminate a target region. The second state may configure the laser light source to perform one or more surgical procedures.

In some cases, each of the plurality of illumination sources may be arranged in a side-by-side or lateral configuration. In such cases, each of the plurality of illumination sources may be separated by a separation distance. The separation distance may be at least about 1 millimeter (mm), 2 mm, 3 mm, 4 mm, 5 mm, 6 mm, 7 mm, 8 mm, 9 mm, 10 mm, or more. In some cases, each of the plurality of illumination sources may be separated by one or more distinct separation distances.

In some cases, each of the plurality of illumination sources may be oriented such that the plurality of illumination sources generates one or more parallel light beams. The one or more parallel light beams may comprise a white light beam generated by the white light source or one or more laser light beams generated by the one or more laser light sources. The one or more parallel light beams may be directed along a path that is perpendicular to a plane corresponding to a position and/or an orientation of a movable plate. In some cases, the one or more parallel light beams may be directed along a path that intersects the plane corresponding to the position and/or orientation of the movable plate at an angle between 0 degrees and 180 degrees.

In some cases, each of the plurality of illumination sources may be oriented such that the plurality of illumination sources generates one or more non-parallel light beams. In such cases, the non-parallel light beams may intersect the plane corresponding to the position and/or orientation of the movable plate at one or more distinct angles. The one or more distinct angles may range from 0 degrees to 180 degrees.

In other cases, each of the plurality of illumination sources may be arranged in a circular configuration or a ring configuration. In such cases, each of the plurality of illumination sources may be positioned around a center point at one or more radial distances. The plurality of illumination sources may be distributed around the center point at one or more angular intervals. The one or more angular intervals may or may not be the same.

In any of the embodiments described herein, the plurality of illumination sources may be arranged such that each of the plurality of illumination sources is disposed at the same distance from the movable plate. In some cases, the plurality of illumination sources may be arranged such that each of the plurality of illumination sources is disposed at one or more distinct distances from a surface or edge of the movable plate.

In any of the embodiments described herein, the plurality of illumination sources may be positioned and/or oriented such that one or more light beams generated by the plurality of illumination sources are directed towards the movable plate along one or more directional vectors. The one or more directional vectors may intersect a surface or an edge of the movable plate at one or more angles. The one or more angles may or may not be distinct. The one or more angles may range from 0 degrees to 360 degrees.

In any of the embodiments described herein, the plurality of illumination sources may be mounted onto a structural component of an illumination module comprising the plurality of illumination sources. The structural component may comprise a wall, a plate, a beam, a rod, or any edge or surface that is internal or external to the illumination module. In some cases, the structural component may be configured to rotate relative to the movable plate.

In some cases, the white light source may be located remote to one or more illumination sources of the plurality of illumination sources (e.g., one or more laser light sources or an ICG excitation light source as described elsewhere herein). In such cases, the white light source may be a third-party light source. The white light source may be configured to generate a white light beam and direct the white light beam to an illumination module comprising one or more laser light sources. The white light beam may be directed towards the illumination module via one or more fiber bundles. The illumination module may then direct the white light beam towards a light aggregation module. The separation of the white light source from the one or more laser light sources may allow the systems disclosed herein to operate with one or more third-party white light sources. Such a configuration may also reduce an amount of heat generated by the plurality of illumination sources and decrease temperature fluctuations while the one or more light sources are operating in an on state (i.e., when the one or more light sources are on and generating one or more light beams). As such, the one or more light beams generated by the plurality of illumination sources may be permitted to stabilize, thereby reducing fluctuations in wavelength and coherence. Further, the separation of the white light source from the one or more laser light sources may minimize a footprint of the illumination module.

In some cases, the illumination module may comprise one or more thermoelectric coolers. The thermoelectric coolers may be configured to cool one or more light sources during operation so that temperature fluctuations may be reduced. As described above, reducing temperature fluctuations may also reduce fluctuations in the wavelength and/or the coherence of the one or more light beams generated by the illumination sources.

In some cases, the plurality of illumination sources may comprise an indocyanine green (ICG) excitation light source. The ICG excitation light source may be configured to generate an ICG excitation light beam. The ICG excitation light beam may cause a fluorescent dye (e.g., indocyanine green) to fluoresce (i.e., emit light). The ICG excitation light beam may have a wavelength of between about 600 nanometers (nm) and about 900 nanometers (nm). The ICG excitation light beam may be emitted onto a target region in a subject's body. The target region may comprise one or more fluorescent dyes configured to absorb the ICG excitation light beam and re-emit fluorescent light with a wavelength between about 750 nanometers (nm) and 950 nanometers (nm). In some cases, the one or more fluorescent dyes may be configured to absorb the ICG excitation light beam and re-emit fluorescent light with a wavelength between about 700 nanometers and 2.5 micrometers (μm). In some cases, the ICG excitation light source may be disposed relative to the movable plate such that the ICG excitation beam does not pass through the movable plate. In other cases, the ICG excitation light source may be disposed relative to the movable plate such that the ICG excitation beam passes through the movable plate.

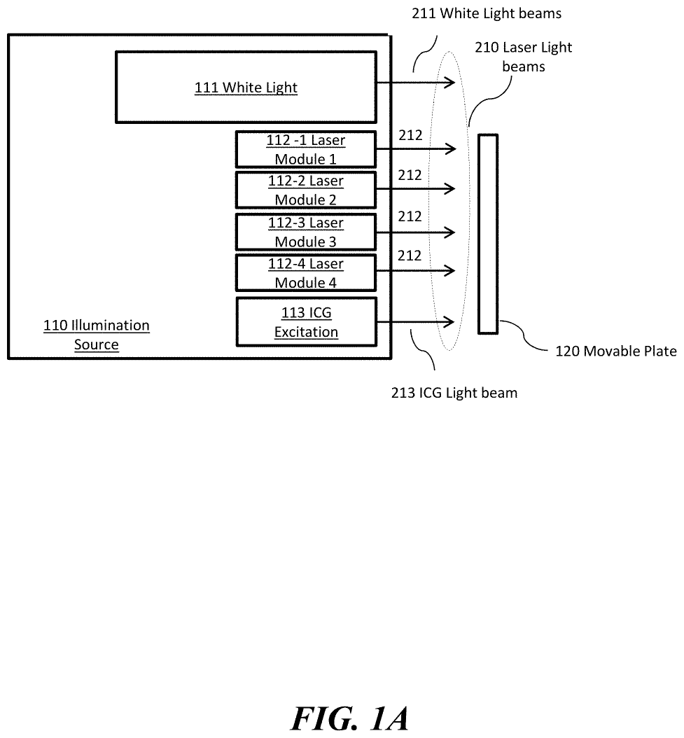

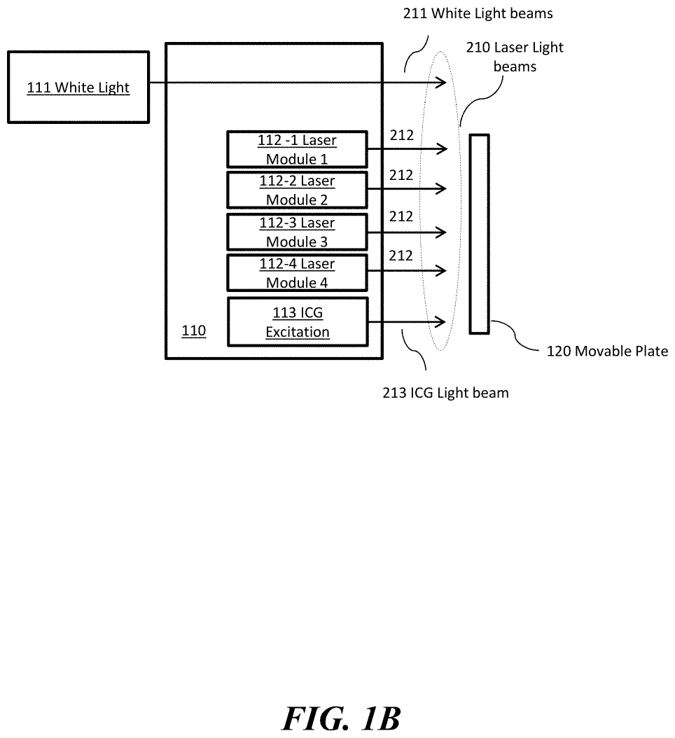

FIGS. 1A-1B illustrate a plurality of illumination sources 110 that may be used to illuminate a target region. The plurality of illumination sources may comprise a white light source 111, one or more laser light sources 112-1, 112-2, 112-3, 112-4, and/or an indocyanine green (ICG) excitation light source 113. The plurality of illumination sources 110 may be configured to generate one or more light beams 210. The one or more light beams 210 may comprise a white light beam 211, one or more laser light beams 212, and/or an ICG excitation light beam 213. The one or more light beams 210 may be directed towards a movable plate 120. As shown in FIG. 1B, in some cases, the white light source 111 may be located remote from an illumination module comprising one or more laser light sources 112-1, 112-2, 112-3, 112-4.

In some cases, the one or more light emitting diodes (LEDs) or laser light sources may comprise two or more LEDs or laser light sources that are configured to generate two or more laser light beams having different wavelengths. In some cases, the two or more laser light sources may be configured to generate two or more laser light beams with a wavelength between about 700 nanometers (nm) and about 1 millimeter (mm). In other cases, the two or more laser light sources may comprise two or more visible light diodes. In such cases, the two or more visible light diodes may be configured to generate two or more laser light beams with a wavelength between about 350 nanometers and about 750 nanometers. In some cases, the two or more laser light beams may have a wavelength between about 400 nanometers and about 700 nanometers.

In some cases, the one or more illumination sources may be configured to generate one or more light pulses. The one or more illumination sources may be configured to generate one or more light pulses using pulse width modulation or pulse duration modulation. A light pulse may be a burst or an emission of light, energy, and/or electrical current. The light pulse may be in the form of an electromagnetic wave. The one or more light pulses may be spaced apart by a predetermined time interval. The one or more light pulses may have a pulse duration. The pulse duration may range between about 1 microsecond to about 100 milliseconds.

In other cases, each of the plurality of illumination sources may be configured to generate one or more continuous light beams. The one or more continuous light beams may be a continuous wave (i.e., a continuous, uninterrupted beam of light with a stable output power). In such cases, a movable plate may be configured to control an exposure of each of the plurality of illumination sources, thereby generating one or more light pulses based on the controlled exposure of the one or more illumination sources.

The plurality of illumination sources may be configured to generate one or more light beams which may be directed towards a movable plate. A movable plate as described herein may be referred to interchangeably as an optical chopper. The movable plate may be a solid object comprising a low transmittance material that is configured to prevent transmission of light through one or more solid portions of the movable plate. The low transmittance material may comprise a dark coating that is configured to prevent transmission of light through one or more solid portions of the movable plate. In some cases, the dark coating may be configured to reduce a radiant power and/or a radiant energy of one or more light beams directed towards the movable plate by a pre-determined amount. In some cases, the pre-determined amount may correspond to a reduction in radiant power and/or radiant energy by at least about 50% or more. The low transmittance material may be sprayed, printed, coated, and/or physically applied onto a surface or an edge of the movable plate. The movable plate may be in the shape of a circle, a square, a rectangle, a triangle, a pentagon, a hexagon, a heptagon, an octagon, a nonagon, a decagon, or any polygon with at least three or more sides. The movable plate may have a horizontal cross-section. The horizontal cross-section may be in the shape of a circle, a square, a rectangle, a triangle, a pentagon, a hexagon, a heptagon, an octagon, a nonagon, a decagon, or any combination of shapes thereof. The movable plate may have a vertical cross-section. The vertical cross-section may be in the shape of a circle, a square, a rectangle, a triangle, a pentagon, a hexagon, a heptagon, an octagon, a nonagon, a decagon, or any combination of shapes thereof.

The movable plate may have one or more dimensions (e.g., height, length, width, and thickness). The one or more dimensions may be at least about 1 millimeter (mm), 2 mm, 3 mm, 4 mm, 5 mm, 6 mm, 7 mm, 8 mm, 9 mm, 10 mm, 10 centimeters (cm), 20 cm, 30 cm, 40 cm, 50 cm, or more.

The movable plate may comprise one or more cut-outs. The one or more cut-outs may correspond to one or more open regions disposed on the movable plate. The one or more open regions may be configured to allow a transmission of light through the movable plate when the one or more cut-outs are optically aligned with at least one of the plurality of illumination sources. In some cases, the one or more open regions may comprise one or more distinct open regions configured to provide one or more distinct exposure times for at least one of the plurality of illumination sources while the movable plate moves (e.g., rotates and/or translates) relative to the plurality of illumination sources. The one or more distinct open regions may have one or more distinct shapes or geometries. The one or more distinct open regions may be disposed on different portions of the movable plate.

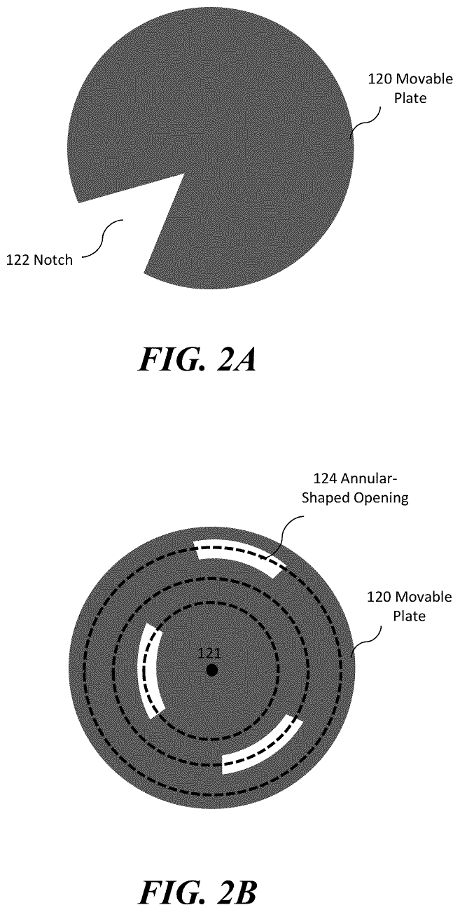

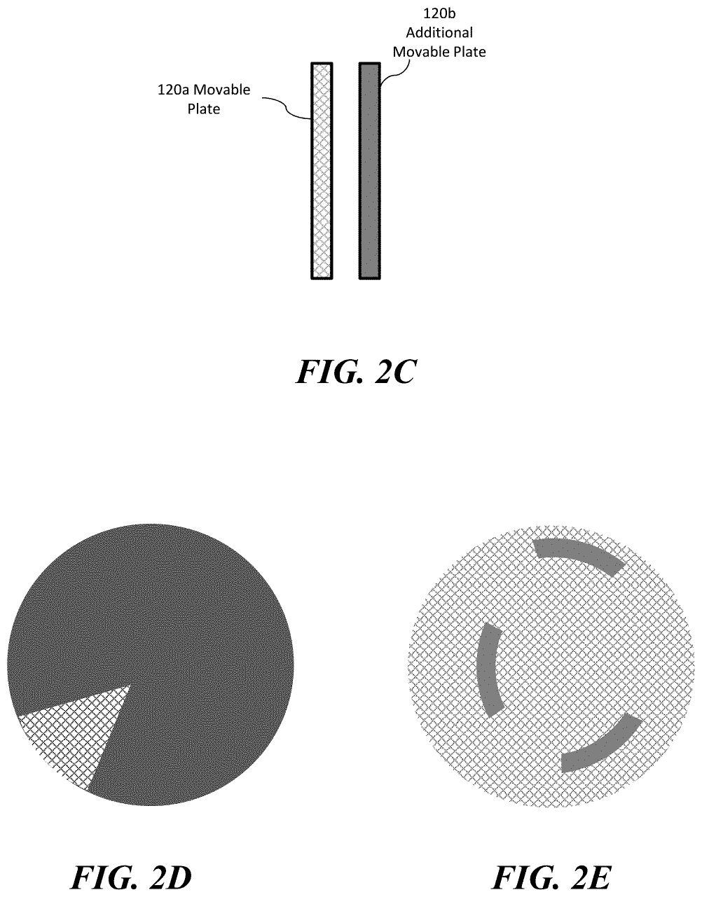

In some cases, the one or more cut-outs may comprise a notch on the movable plate. FIG. 2A illustrates a movable plate 120 comprising a notch 122. The notch 122 may be an indentation or an incision on an edge or a surface of the movable plate 120. The notch 122 may be in the shape of a triangle, a wedge, or a circular sector (i.e., a portion of a disk enclosed by two radii and an arc). The notch 122 may span a portion of a length, width, height, or circumference of the movable plate. The notch 122 may span an angular range that is greater than 0 degrees and less than 360 degrees. In some cases, the one or more cut-outs of the movable plate 120 may comprise a plurality of notches 122 arranged on different portions or sections of the movable plate 120.

In some cases, the one or more cut-outs may comprise one or more annular-shaped openings on the movable plate. FIG. 2B illustrates a movable plate 120 with one or more annular-shaped openings 124. The one or more annular-shaped openings 124 may be disposed at different radial distances from a center 121 of the movable plate 120. The one or more annular-shaped openings may have an annular shape corresponding to a ring or a portion of a ring. The annular shape may be a shape bounded by two concentric circles. The two concentric circles may or may not have a center that corresponds with a center of the movable plate. Alternatively, the annular shape may be a shape bounded by two concentric arcs and two edges. The two edges may or may not coincide with a radial line extending from a center point associated with the two concentric arcs.

In some cases, the one or more open regions disposed on the movable plate may comprise one or more annular-shaped openings disposed at one or more radial distances from a center of the movable plate. The one or more radial distances may be distinct. In such cases, each of the one or more radial distances may correspond respectively to at least one of the plurality of illumination sources. The one or more annular-shaped openings may be disposed at one or more distinct angular positions relative to each other. In some cases, the one or more annular-shaped openings may be disposed at the same radial distance from the center of the movable plate.

In some cases, the one or more open regions may comprise a first annular-shaped opening and a second annular-shaped opening. In such cases, the first annular-shaped opening may have a first circumferential length that is different than a second circumferential length of the second annular-shaped opening. In other cases, the one or more open regions may comprise three or more annular-shaped openings with different circumferential lengths. The circumferential length may be configured to provide a pre-determined exposure time for each of said plurality of illumination sources. The pre-determined exposure time may be determined based on the circumferential length and/or a rate of rotation (i.e., a rotational rate) of the movable plate.

In some cases, the one or more open regions may comprise one or more wedge-shaped openings. The one or more wedge-shaped openings may be disposed at one or more distinct angular positions relative to each other. In some cases, the one or more open regions may comprise a first wedge-shaped opening and a second wedge-shaped opening. The first wedge-shaped opening may have a first circumferential width that is different than a second circumferential width of a second wedge-shaped opening. In some cases, the one or more open regions may comprise three or more wedge-shaped openings with different circumferential widths.

As described above, the one or more wedge-shaped openings may have a circumferential width. The circumferential width may correspond to a portion of a length of an edge of the movable plate. The circumferential width may be configured to provide a pre-determined exposure time for each of said plurality of illumination sources. The pre-determined exposure time may be determined based on the circumferential width and/or a rotational rate of the movable plate.

In some cases, the one or more distinct open regions may comprise (i) a first open region configured to expose at least one of the plurality of illumination sources for a first pre-determined time interval, and (ii) a second open region configured to expose at least one of the plurality of illumination sources for a second pre-determined time interval. The first open region may have a different geometry and/or shape than the second open region. The first pre-determined time interval may be different than the second pre-determined time interval. In some cases, the one or more distinct open regions may comprise three or more open regions configured to expose each of the plurality of illumination sources for one or more distinct pre-determined time intervals.

The movable plate and/or the one or more cut-outs of the movable plate may be optically aligned with one or more of the plurality of illumination sources. The movable plate and/or the one or more cut-outs of the movable plate may be optically aligned with an illumination source when the illumination source (e.g., a white light source, laser light source, or indocyanine (ICG) excitation light source) is positioned and/or oriented relative to the movable plate such that one or more light beams generated by the illumination source are emitted and/or transmitted along a light path or vector that intersects and/or coincides with (a) the movable plate or (b) an area corresponding to a cut-out portion of the movable plate.

The movable plate may be configured to move (e.g., rotate or translate) relative to the one or more illumination sources to successively (a) allow a transmission of light through one or more cut-outs, and (b) inhibit a transmission of light by physically blocking such transmission of light with one or more solid portions of the movable plate. The one or more solid portions may comprise a low transmittance material, as described elsewhere herein.

The movable plate may be configured to rotate in a clockwise direction and/or a counter-clockwise direction. The movable plate may be configured to rotate at a pre-determined rotational rate. The pre-determined rotational rate may be at least about 100 rotations per minute (RPM), 200 RPM, 300 RPM, 400 RPM, 500 RPM, 600 RPM, 700 RPM, 800 RPM, 900 RPM, 1000 RPM, 1100 RPM, 1200 RPM, 1300 RPM, 1400 RPM, 1500 RPM, 1600 RPM, 1700 RPM, 1800 RPM, 1900 RPM, 2000 RPM, or more.

The movable plate may be configured to control an exposure of the one or more illumination sources by selectively allowing one or more light beams generated by the one or more illumination sources to pass through the one or more cut-outs of the moveable plate, during one or more pre-determined time intervals. The one or more pre-determined time intervals may be determined based on (i) a rotational rate of the movable plate and/or (ii) a shape or geometry associated with the one or more cut-outs. During the one or more pre-determined time intervals, at least one of the plurality of illumination sources may be optically aligned with at least one of the one or more cut-outs.

The movable plate may be configured to control the exposure of the one or more illumination sources relative to a pre-determined frame capture rate. In some cases, the movable plate may be configured to rotate at a pre-determined rate of rotation such that at least a subset of the plurality of illumination sources is exposed for one or more time intervals corresponding to an imaging period. The imaging period may correspond to one or more time intervals during which an imaging device with the pre-determined frame capture rate is configured to acquire one or more image frames. The imaging device may comprise an image sensor or a camera.

The movable plate may be configured to generate one or more light pulses based on the controlled exposure of the one or more illumination sources. The one or more light pulses may be obtained from one or more light beams (e.g., a white light beam, a laser light beam, and/or an indocyanine green (ICG) excitation light beam) generated by the plurality of illumination sources. A light pulse may be generated when the movable plate translates or rotates between a first position that optically aligns one or more illumination sources with one or more cut-outs, and a second position that optically aligns the one or more illumination sources with a solid portion of the movable plate. The pulse duration associated with a light pulse may correspond to a time period during which the movable plate translates or rotates between the first position and the second position. The pulse duration may be a function of the shape or geometry of the movable plate and/or the shape or geometry of the one or more cut-outs. For example, a first cut-out with a first dimension may produce a first light pulse with a first pulse duration that is longer than a second pulse duration associated with a second light pulse produced by a second cut-out with a second dimension that is less than the first dimension. The pulse duration may be a function of a rate of rotation of the movable plate. For example, the pulse duration may be longer when the movable plate rotates at a lower rate of rotation since a lower rate of rotation may allow for an illumination source to be optically aligned with one or more cut-outs for a longer period of time.

The use of the movable plate to generate one or more light pulses may permit the systems disclosed herein to operate with the plurality of illumination sources continuously on during medical imaging, and without electronically pulsing the illumination sources. This may allow the plurality of illumination sources to generate and maintain one or more coherent light beams. As an additional advantage, the one or more light beams (e.g., white light beam, laser light beam, and/or ICG excitation light beam) may be permitted to stabilize such that the coherence of the one or more light beams is improved relative to a system where the illumination sources are pulsed electronically, or where the illumination sources are alternated between an on state and an off state.

The exposure of each of the plurality of illumination sources may be synchronized to an image frame capture rate associated with an imaging device (e.g., an image sensor and/or a camera). As such, the generation and/or transmission of one or more light pulses may be synchronized to the acquisition of one or more image frames captured by an imaging device. The one or more image frames may be acquired at an image frame capture rate associated with the imaging device. The one or more image frames captured by the imaging device may comprise spectral data generated in part based on the interaction (i.e., a reflection and/or a deflection) of a subset of the one or more light pulses with a target region in a subject's body. In some cases, each of the one or more image frames may correspond to a different illumination source of the plurality of illumination sources, or a different subset of the plurality of illumination sources.

In some cases, the exposure of each of the plurality of illumination sources may be synchronized to an image frame capture rate of an imaging device using a timing signal. The timing signal may be generated using one or more photointerrupters. A photointerrupter may comprise a sensor configured to detect if one or more light beams are incident on the sensor. In some cases, one or more photointerrupters may be positioned adjacent to each of the plurality of illumination sources. The one or more photointerrupters may be configured to generate a timing signal, which may be provided to a microcontroller configured to (i) adjust the image frame capture rate of the imaging device and/or (ii) adjust a time at which the camera captures one or more image frames. Adjusting the image frame capture rate may involve synchronizing the image frame capture rate to a rate at which one or more illumination sources are exposed through a notch of the movable plate. Alternatively, adjusting the image frame capture rate may involve synchronizing the image frame capture rate to a time at which one or more illumination sources are exposed through a notch of the movable plate. In some cases, the microcontroller may be configured to compensate for any delays associated with transmission of the timing signal by modifying the timing signal.

In some cases, the timing signal generated by the one or more photointerrupters may be provided to a microcontroller, a field-programmable gate array (FPGA), or one or more electronic gates. The microcontroller, the field-programmable gate array (FPGA), and/or the one or more electronic gates may be configured to generate a trigger signal based on the timing signal obtained from the one or more photointerrupters. The trigger signal may be used by the camera or the imaging device to trigger an exposure of one or more image frames.

In some embodiments, the medical imaging system may comprise a movable plate with a cut-out, three laser diodes, and three photointerrupters located adjacent to the three laser diodes. The movable plate may be configured to rotate relative to the three laser diodes at a rotational rate of 2400 rotations per minute (RPM). In such cases, the three photointerrupters may be configured to pulse at 40 hertz (Hz). The medical imaging system may comprise a microcontroller, a field-programmable gate array (FPGA), and/or one or more electronic gates configured to combine one or more pulses generated by the three photointerrupters into a trigger signal at 120 Hz. The camera or imaging device may be configured to capture one or more image frames upon receiving the trigger signal. The camera or imaging device may be configured to acquire one or more image frames at 120 frames per second. In such embodiments, a commanded speed of a motor that is configured to control a movement of the movable plate may dictate an image frame capture rate of the camera or imaging device.

In some cases, the exposure of each of the plurality of illumination sources may be synchronized to an image frame rate of an imaging device using a timing signal generated by the imaging device. In such cases, the imaging device may be configured to generate a timing signal based on a rate at which the camera captures one or more image frames and/or a time at which the camera captures one or more image frames. The timing signal may be provided to a microcontroller, which may be configured to adjust a rate and/or a time at which one or more illumination sources are pulsed or exposed through a notch of the movable plate. In some cases, adjusting the rate of exposure of the one or more illumination sources may involve adjusting a speed at which the movable plate rotates or translates relative to the one or more illumination sources. In other cases, adjusting the rate of exposure of the one or more illumination sources may involve adjusting a time at which one or more notches of the movable plate are optically aligned with the one or more illumination sources.

In any of the embodiments disclosed herein, the system may further comprise an additional movable plate configured to rotate relative to the plurality of illumination sources and the movable plate. The additional movable plate may be configured to rotate at a second rate that is different than a first rate at which the movable plate is configured to rotate. The additional movable plate may be configured to rotate in a second direction that is different than a first direction in which the movable plate is configured to rotate. The movable plate may comprise a first set of cut-outs with a different geometry or arrangement than a second set of cutouts on the additional movable plate. The movable plate may have a first shape or geometry that is a different than a second shape or geometry of the additional movable plate. The movable plate and the additional movable plate may have different shapes, geometries, and/or dimensions.

FIG. 2C illustrates a movable plate 120a and an additional movable plate 120b. The movable plate 120a and the additional movable plate 120b may be configured to control an exposure of each of the plurality of illumination sources. As described above, the movable plate 120a may be configured to rotate in a different direction and/or at a different rate of rotation than the additional movable plate 120b.

FIG. 2D and FIG. 2E illustrate a top view and a bottom view of the movable plate 120a and the additional movable plate 120b when the respective centers of the movable plate 120a and the additional movable plate 120b are aligned (i.e., lie on a similar axis). As shown in FIG. 2D and FIG. 2E, the movable plate 120a may comprise a first set of cut-outs with a different geometry or arrangement than a second set of cut-outs on the additional movable plate 120b. In such cases, the movable plate 120a and the additional movable plate 120b may be used simultaneously to modulate the length and/or timing of the exposure of the illumination sources in a different manner than if only one movable plate (e.g., either the movable plate 120a or the additional movable plate 120b) were used to modulate the exposure of the illumination sources.

In some embodiments, the medical imaging systems of the present disclosure may comprise one or more optical isolators. An optical isolator may comprise an optical device or component that only allows unidirectional transmission of an optical signal (e.g., one or more light beams or light pulses generated using one or more illumination sources as described herein). The optical isolator may be used to create a more stable coherent light source for imaging. The optical isolator may also be used to avoid unwanted optical reflections and to minimize external optical feedback (e.g., back reflections) that can damage the one or more illumination sources and/or cause instability. The optical isolator may comprise a polarization dependent isolator. Alternatively, the optical isolator may comprise a polarization independent isolator.

In some cases, the optical isolator may be integrated with the one or more illumination sources described elsewhere herein. In such cases, the optical isolator may be disposed on a portion or a structural component of the one or more illumination sources, and may be positioned along a beam path of the one or more illumination sources. In other cases, the optical isolator may be integrated with the movable plate (i.e., the optical chopper) described elsewhere herein. Alternatively, the optical isolator may be disposed between the one or more illumination sources and the movable plate, along a beam path of the one or more illumination sources.

In some embodiments, the medical imaging systems of the present disclosure may comprise one or more bandpass filters. The one or more bandpass filters may be used in combination with any of the illumination sources described herein (e.g., the white light source, the laser light source, the ICG excitation light source, etc.). In some embodiments, the one or more bandpass filters may be used on the ICG excitation lasers to create a narrower laser source around an 808 nanometer (nm) excitation wavelength so that the 808 nm excitation wavelength can be effectively blocked with a notch filter, thereby allowing visualization of only the features in a target region that fluoresce in response to the excitation wavelength.

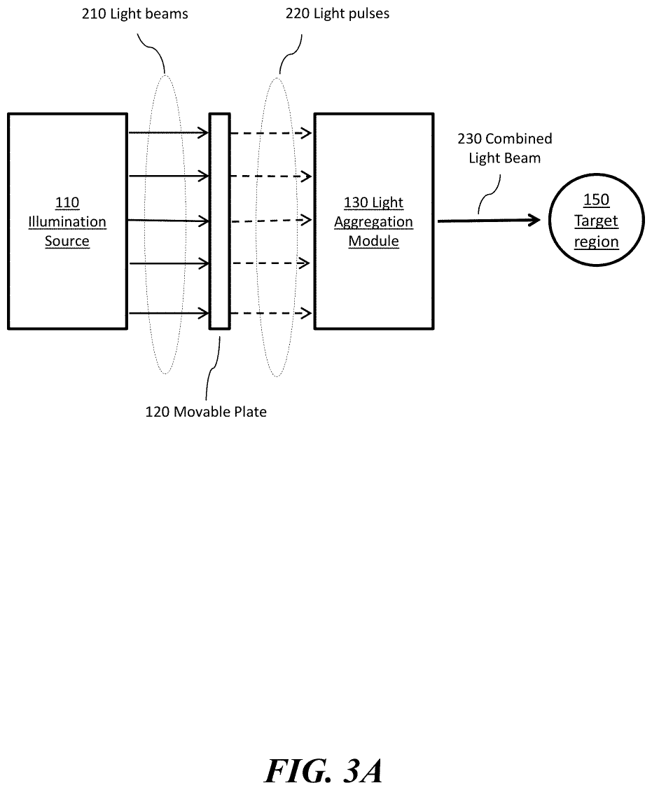

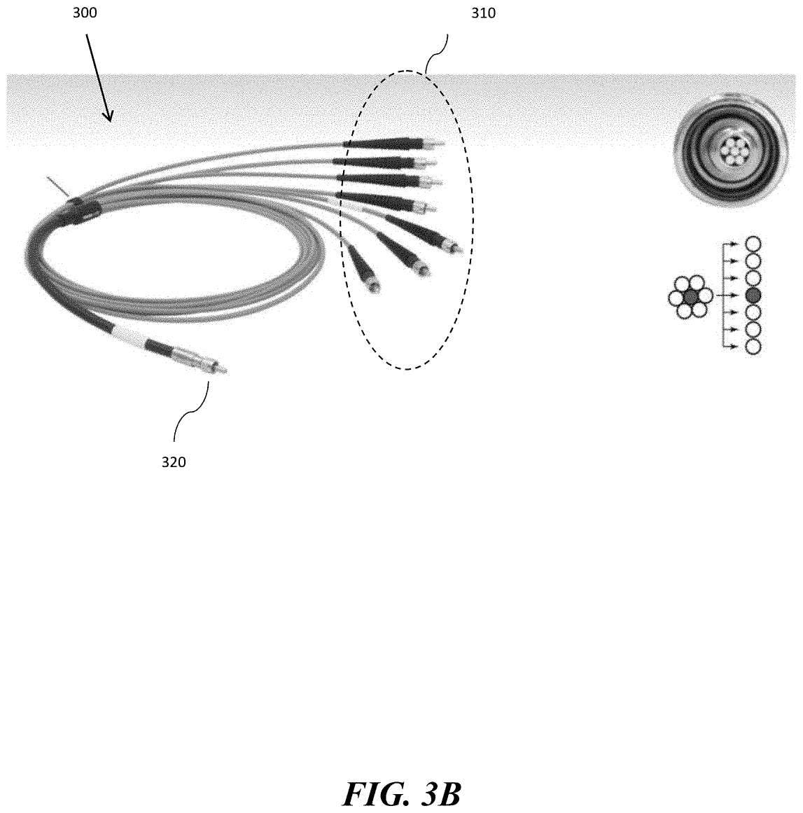

FIG. 3A illustrates a system for illuminating a target region in a subject's body. The system may comprise a plurality of illumination sources 110. The plurality of illumination sources 110 may be configured to generate one or more light beams 210. The one or more light beams 210 may be directed towards a movable plate 120. In some cases, a subset of the one or more light beams 210 may be directed towards the movable plate 120. The movable plate 120 may be configured to generate one or more light pulses 220 by selectively controlling the exposure of the one or more illumination sources 110 such that one or more light beams 210 are permitted to pass through a cut-out of the movable plate 120 during one or more discrete time intervals. During the one or more discrete time intervals, a subset of the plurality of illumination sources 110 may be optically aligned with a cut-out of the movable plate 120.

The one or more light pulses 220 generated by the movable plate 120 may be aggregated by a light aggregation module 130. The light aggregation module 130 may be configured to receive the one or more light pulses 220. The one or more light pulses 220 may be provided to the light aggregation module 130 via one or more optical fiber bundles. The one or more optical fiber bundles may be configured to receive multiple signals (e.g., light beams 210 or light pulses 220) from multiple illumination sources 110 via multiple separate fibers.

The aggregation of one or more light pulses may be performed using a bifurcated fiber bundle. FIG. 3B illustrates an example of a bifurcated fiber bundle 300. The bifurcated fiber bundle 300 may comprise a plurality of separate fiber bundles 310 at a first end of the bifurcated fiber bundle 300. Each of the separate fiber bundles 310 at the first end of the bifurcated fiber bundle 300 may be configured to receive one or more light pulses generated using the movable plate and the plurality of illumination sources. The light pulses generated by pulsing each of the plurality of illumination sources may be collimated into one or more fiber bundles of the separate fiber bundles 310. In some cases, one or more light pulses generated using a white light source may also be coupled into a fiber bundle of the separate fiber bundles 310. The separate fiber bundles 310 may be combined and packed into a single fiber bundle 320 located at a second end of the bifurcated fiber bundle 300. The separate fiber bundles 310 may be configured to direct one or more light pulses from a first end of the bifurcated fiber bundle 300 to the single fiber bundle 320 located at the second end of the bifurcated fiber bundle 300. The single fiber bundle 320 may be configured to aggregate and direct the one or more light pulses generated using the movable plate and the plurality of illumination sources to a scope. The single fiber bundle 320 may be directly or indirectly coupled to the scope. The bifurcated fiber bundle 300 may be an N to 1 bifurcated fiber bundle, wherein N is an integer that corresponds to a number of illumination sources within the plurality of illumination sources. N may be at least about 2, 3, 4, 5, 6, 7, 8, 9, 10, or more. In some cases, the bifurcated fiber bundle 300 may be a 5 to 1 bifurcated fiber bundle.

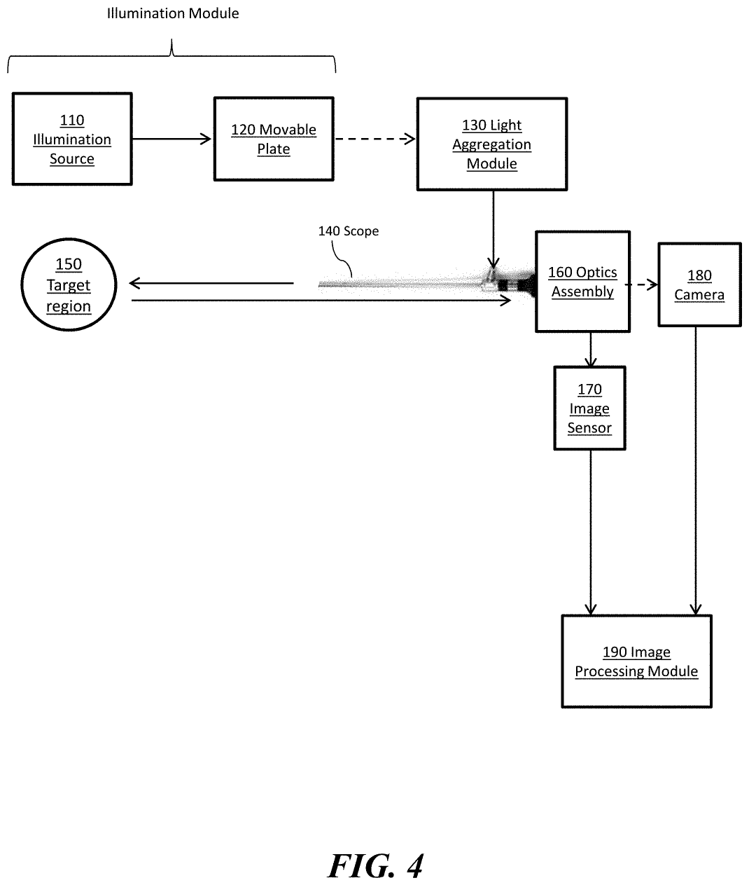

As shown in FIG. 3A and FIG. 4, the light aggregation module 130 may be configured to generate a combined beam 230 based on (a) one or more light beams 210 generated by the plurality of illumination sources and/or (b) one or more light pulses 220 generated by movable plate 120. The combined light beam 230 may be provided to a scope 140. The scope 140 may comprise a laparoscope, an endoscope, a borescope, a videoscope, or a fiberscope. The scope 140 may be insertable into the subject's body and configured to direct the combined light beam 230 onto the target region 150. The scope may be configured to receive, at a distal end of the scope, a reflected light signal that is generated when the combined light signal 230 is emitted onto and reflected from the target region 150. The reflected light signal may comprise a first portion of the reflected light signal and a second portion of the reflected light signal. The first portion of the reflected light signal may comprise deflected light (e.g., backscattered light) that is generated when the target site is illuminated with a first illumination source (e.g., a white light source, a laser light source, or an indocyanine green (ICG) excitation light source). The second portion of the reflected light signal may comprise reflected light that is generated when the target site is illuminated with a second illumination source that is different than the first illumination source.

As shown in FIG. 4, the scope may be configured to direct the reflected light signal from a distal end of the scope 140 to a proximal end of the scope 140 and into an optics assembly 160 located adjacent to the proximal end of the scope. The optics assembly 160 may comprise a beam splitter. The reflected light signal may pass through the beam splitter (e.g., a dichroic mirror). In doing so, the reflected light signal may be separated into the first portion of the reflected light signal and the second portion of the reflected light signal. The first portion may be provided to an image sensor 170 to generate a first image. The second portion may be provided to a camera 180 to generate a second image. The image sensor 170 may be configured to provide the first image to an image processing module 190. The camera 180 may be configured to provide the second image to the image processing module 190. The image processing module 190 may be configured to generate a combined image based on the first image and the second image. The combined image may be an overlaid or superimposed image comprising one or more features from the first image and one or more features from the second image.

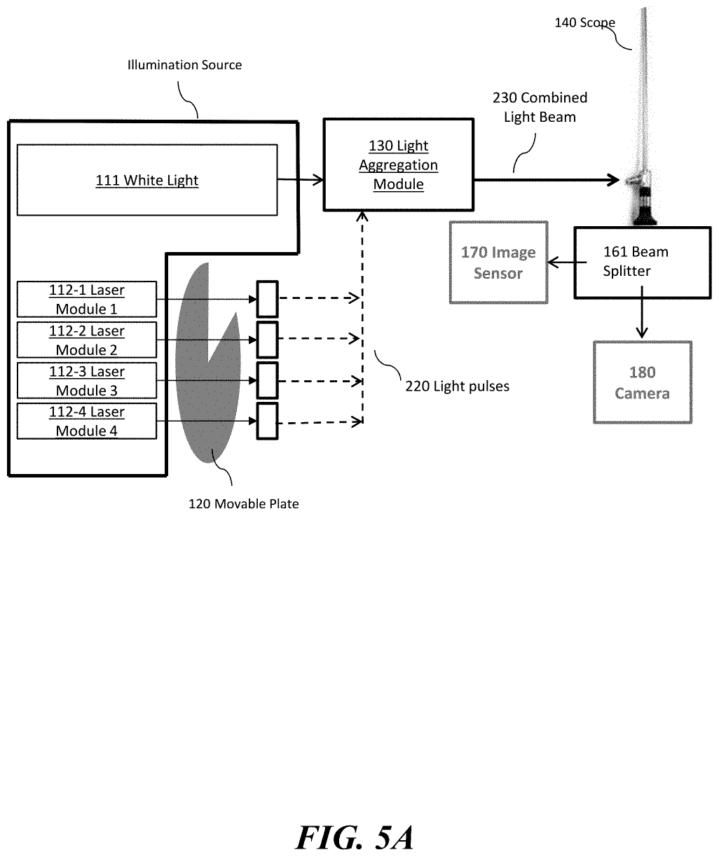

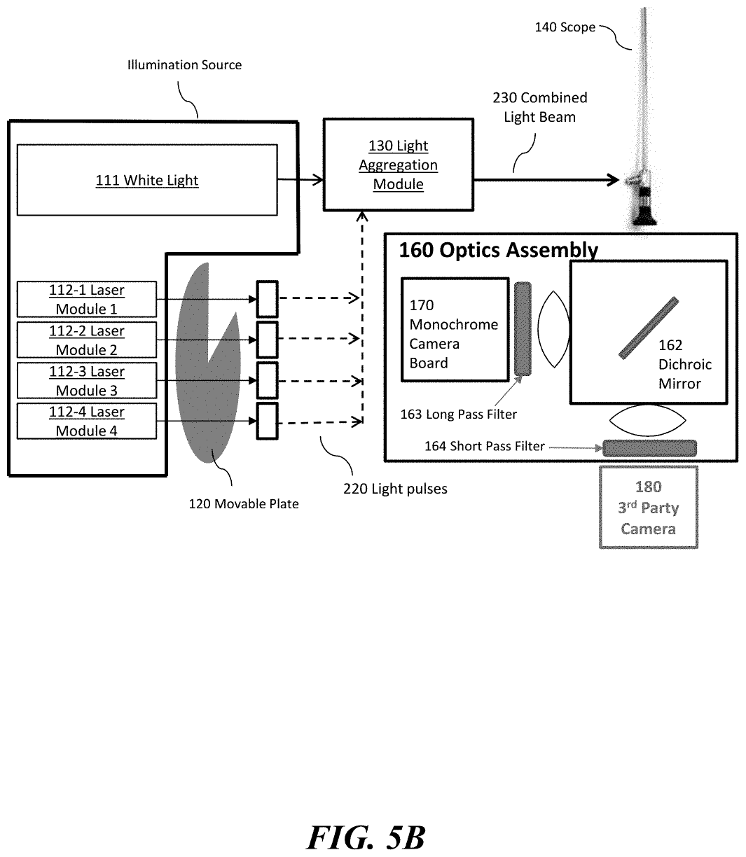

As illustrated in FIGS. 5A and 5B, in some cases, the movable plate 120 may be optically aligned with one or more laser light sources 112-1, 112-2, 112-3, and 112-4. The plurality of illumination sources may comprise the white light source 111 and the one or more laser light sources 112-1, 112-2, 112-3, and 112-4. In such cases, the movable plate 120 and the white light source 111 may not share a common optical axis (i.e., the white light source may be positioned and/or oriented such that a white light beam generated by the white light source does not intersect or coincide with either the movable plate or a cut-out portion of the movable plate). The white light source may be disposed relative to the movable plate such that the white light beam does not pass through the movable plate. The white light beam from the white light source may be transmitted continuously without being affected or separated into pulses by the movable plate. The one or more light pulses generated by the movable plate may be obtained from the one or more laser light beams.

In such cases, the light aggregation module 130 may be configured to (i) combine (a) the one or more light pulses 220 obtained from the one or more laser light beams with (b) the white light beam to generate (c) a combined light beam 230, and (ii) provide the combined light beam 230 to a scope 140, which scope may be insertable into the subject's body and configured to direct the combined light beam 230 onto a target region inside a subject's body.

In any of the embodiments described herein, the light aggregation module may be configured to aggregate two or more light signals. The two or more light signals may comprise light pulses and/or light beams. Aggregating the two or more light signals may involve (i) combining two or more light pulses, (ii) combining two or more light beams, and/or (iii) combining one or more light pulses with one or more light beams. Aggregating the two or more light signals may involve one or more aspects of spectral beam combining. Spectral beam combining may involve combining two or more incoherent signals with non-overlapping optical spectra using a wavelength-sensitive beam combiner (e.g., a prism, a diffraction grating, a dichroic mirror, and/or a volume Bragg grating) that can deflect incident signals (i.e., pulses or beam) according to their respective wavelengths, so that these signals all propagate in the same direction. In some cases, spectral beam combining may be performed using a series of dichroic mirrors configured to reflect a plurality of light beams and/or light pulses along one or more beam paths that may coincide with each other. In such cases, the light beams and/or light pulses may propagate in the same direction.

In some cases, the light aggregation module may be configured to combine a first set of light pulses with a second set of light pulses. In such cases, combining the first set of light pulses and the second set of light pulses may involve sequentially aligning one or more light pulses from either the first set or the second set of light pulses, in a temporal manner. Alternatively, combining the first set of light pulses and the second set of light pulses may involve one or more aspects of coherent beam combining and/or spectral beam combining.

In other cases, the light aggregation module may be configured to combine a first set of light beams with a second set of light beams. In such cases, combining a first set of light beams with a second set of light beams may involve one or more aspects of coherent beam combining and/or spectral beam combining.

As described elsewhere herein, a reflected light signal may be generated when the combined light signal 230 is emitted onto and reflected from the target region. The reflected light signal may comprise a first portion of the reflected light signal and a second portion of the reflected light signal. The first portion of the reflected light signal may comprise deflected light (e.g., backscattered light) that is generated when the target site is illuminated with one or more light pulses obtained from one or more coherent laser light beams generated by the one or more laser light sources. The second portion of the reflected light signal may comprise reflected light that is generated when the target site is illuminated with a white light beam generated by the white light source. The scope 140 may be configured to direct the reflected light signal towards a beam splitter 161. The beam splitter may be configured to separate the reflected light signal into the first portion of the reflected light signal and the second portion of the reflected light signal. The first portion may be provided to an image sensor 170 to generate a first image. The second portion may be provided to a camera 180 to generate a second image. The image sensor 170 may be configured to provide the first image to an image processing module. The camera 180 may be configured to provide the second image to the image processing module. The image processing module may be configured to generate a combined image based on the first image and the second image.

FIG. 5B illustrates an optics assembly 160 that may be configured to receive the reflected light signal. The optics assembly 160 may comprise a dichroic mirror 162. In some cases, the dichroic mirror may be interchanged with a beam splitter, a half mirror, a dichroic beam splitter, or a multi-band beam splitter. The dichroic mirror 162 may be configured to receive the reflected light signal from the target site and (i) reflect the first portion of the light signals that is in a first electromagnetic spectral range toward an image sensor 170, and (ii) permit the second portion of the light signals in a second electromagnetic spectral range to pass through towards a camera 180. The camera 180 may or may not be integrated with the optics assembly 160. The optics assembly 160 may comprise a long pass filter 163. The long pass filter 163 may be positioned adjacent to and/or in front of the image sensor 170. The image sensor may be a monochrome camera board. The optics assembly 160 may comprise a short pass filter 164. The short pass filter 164 may be positioned adjacent to and/or in front of the camera 180.

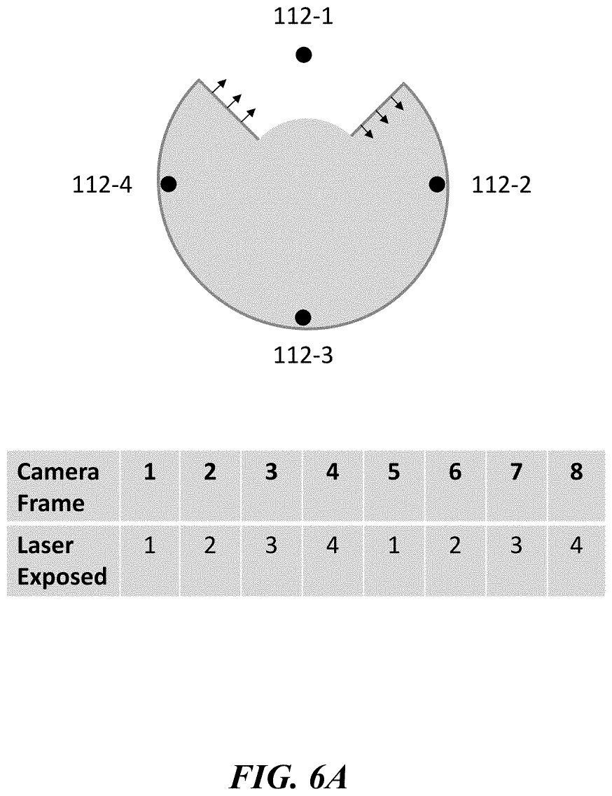

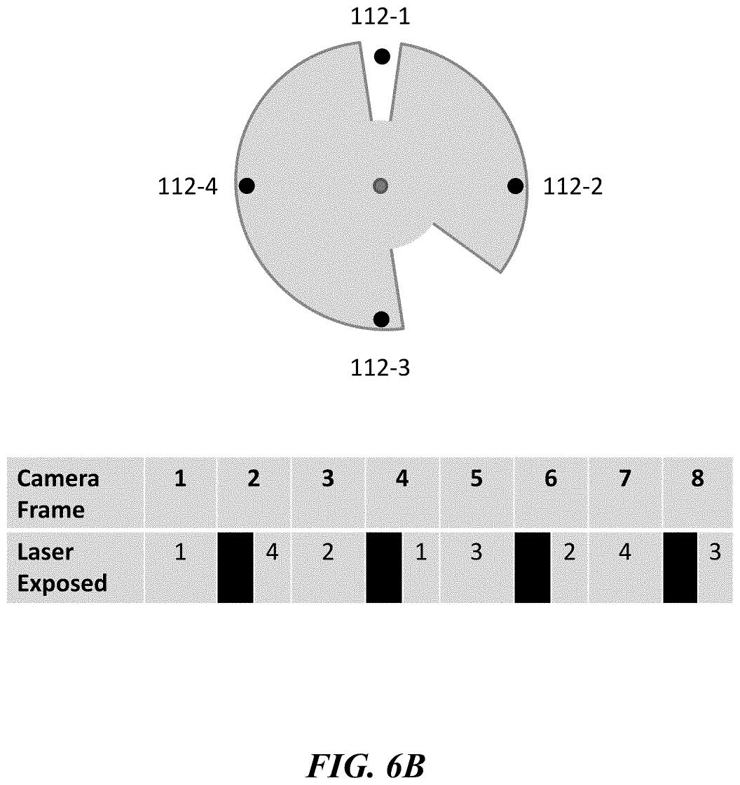

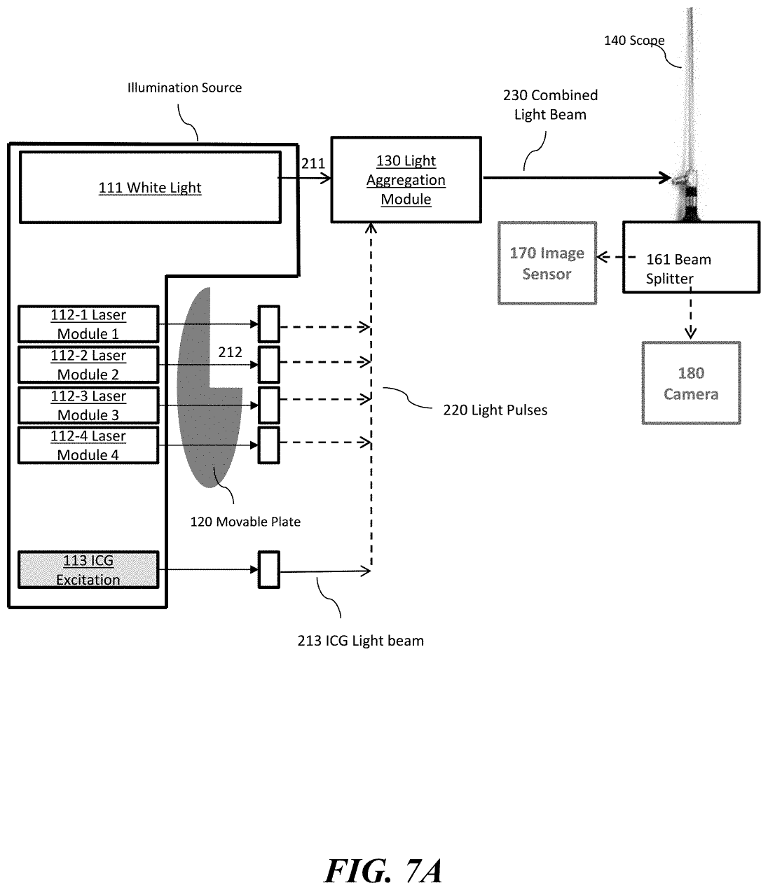

FIG. 6A illustrates a synchronization of (i) the exposure of one or more laser light sources 112-1, 112-2, 112-3, and 112-4 through the movable plate 120 with (ii) one or more camera frames captured by the image sensor or the camera. The movable plate 120 may comprise a single notch. The camera frames may be acquired at 120 frames per second (FPS). Each laser light source 112-1, 112-2, 112-3, and 112-4 may be exposed at a frequency of 30 hertz (Hz). In such cases, the movable plate 120 may rotate at about 1800 rotations per minutes (RPM).

FIG. 6B illustrates a synchronization of (i) the exposure of one or more laser light sources 112-1, 112-2, 112-3, and 112-4 through the movable plate 120 with (ii) one or more camera frames captured by the image sensor or the camera. The movable plate 120 may comprise two distinct notches with different shapes and/or geometries. Each of the two distinct notches may provide different exposure times for each of the laser light sources 112-1, 112-2, 112-3, and 112-4. The camera frames may be acquired at 120 frames per second (FPS). For each revolution of the movable plate, each laser light source 112-1, 112-2, 112-3, and 112-4 may be (i) exposed through a first notch of the movable plate for a first exposure time and (ii) a second notch of the movable plate for a second exposure time. The movable plate 120 may be configured to rotate at about 900 rotations per minutes (RPM).

In some cases, the movable plate may be optically aligned with both (i) the white light source and (ii) the one or more laser light sources. The plurality of illumination sources may comprise the white light source and the one or more laser light sources. In such cases, the one or more light pulses generated by the movable plate may be obtained from (i) the white light beam and (ii) the one or more laser light beams.

In such cases, the light aggregation module may be configured to (i) combine (a) the one or more light pulses obtained from the white light beam with (b) the one or more light pulses obtained from the one or more laser light beams, to generate (c) a combined light beam, and (ii) provide the combined light beam to a scope. The scope may be insertable into the subject's body and configured to direct the combined light beam onto the target region.

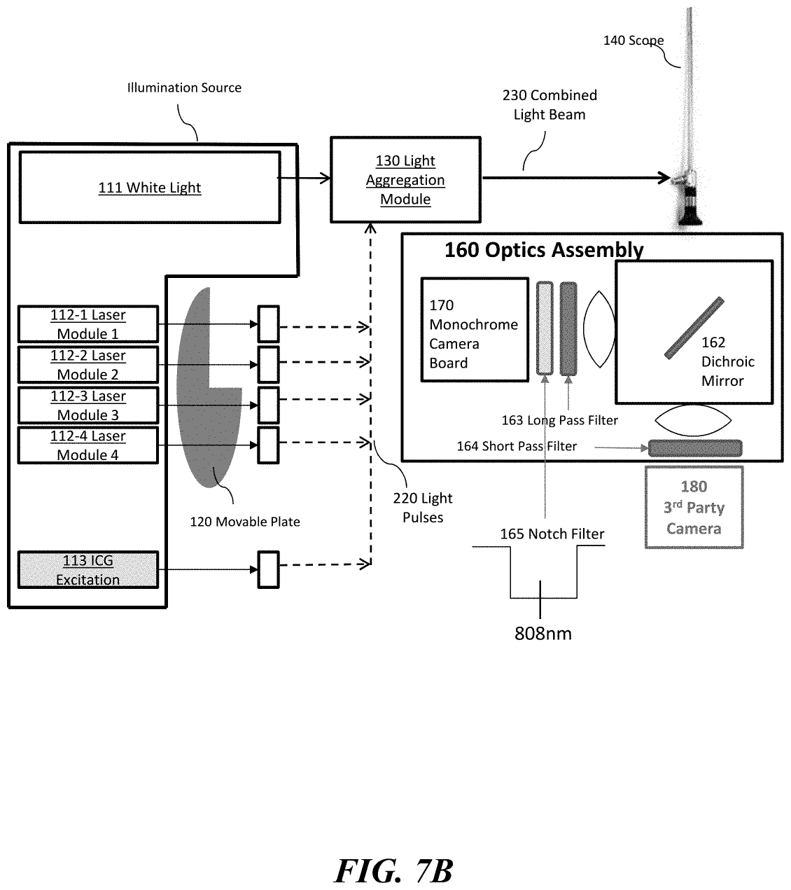

As illustrated in FIGS. 7A-7B, in some cases, the movable plate 120 may be optically aligned with (i) the one or more laser light sources 112-1, 112-2, 112-3, and 112-4. The plurality of illumination sources may comprise the white light source 111, the one or more laser light sources 112-1, 112-2, 112-3, and 112-4, and the ICG excitation light source 113. In such cases, the movable plate 120 and the white light source 111 may not share a common optical axis. The white light beam from the white light source may be transmitted continuously without being affected or separated into pulses by the movable plate. Further, the movable plate 120 and the ICG excitation light source 113 may not share a common optical axis. The ICG excitation light beam 213 from the ICG excitation light source 113 may be transmitted continuously without being affected or separated into pulses by the movable plate 120. The one or more light pulses 220 generated by the movable plate 120 may be obtained from (i) the one or more laser light beams 212.

In such cases, the light aggregation module 130 may be configured to (i) combine (a) the one or more light pulses 220 obtained from the one or more laser light beams 212 with (b) at least one of the white light beam 211 or the ICG excitation light beam 213 to generate (c) a combined light beam 230, and (ii) provide the combined light beam 230 to a scope 140. The scope may be insertable into the subject's body and configured to direct the combined light beam onto a target region within the subject's body.

As described above, a reflected light signal may be generated when the combined light signal 230 is emitted onto and reflected from the target region. The reflected light signal may comprise a first portion of the reflected light signal and a second portion of the reflected light signal. The first portion of the reflected light signal may comprise deflected light that is generated when the target site is illuminated with one or more light pulses obtained from the one or more laser light beams 212. The second portion of the reflected light signal may comprise reflected light that is generated when the target site is illuminated with a different light (e.g., a white light beam 211 or an ICG excitation light beam 213). The scope 140 may be configured to direct the reflected light signal towards a beam splitter 161. The beam splitter may be configured to separate the reflected light signal into the first portion of the reflected light signal and the second portion of the reflected light signal. The first portion may be provided to an image sensor 170 to generate a first image. The second portion may be provided to a camera 180 to generate a second image. The image sensor 170 may be configured to provide the first image to an image processing module. The camera 180 may be configured to provide the second image to the image processing module. The image processing module may be configured to generate a combined image based in part on the first image and/or the second image.

FIG. 7B illustrates an optics assembly 160 that may be configured to receive the reflected light signal. The optics assembly 160 may comprise a dichroic mirror 162. In some cases, the dichroic mirror may be interchanged with a beam splitter, a half mirror, a dichroic beam splitter, or a multi-band beam splitter. The dichroic mirror 162 may be configured to receive the reflected light signal from the target site and (i) reflect the first portion of the light signals that is in a first electromagnetic spectral range toward an image sensor 170, and (ii) permit the second portion of the light signals in a second electromagnetic spectral range to pass through toward a camera 180. The camera 180 may or may not be integrated with the optics assembly 160. The optics assembly 160 may comprise a long pass filter 163. The long pass filter 163 may be positioned adjacent to and/or in front of the image sensor 170. The image sensor may be a monochrome camera board. The optics assembly 160 may comprise a short pass filter 164. The short pass filter 164 may be positioned adjacent to and/or in front of the camera 180. The optics assembly may comprise a notch filter 165. The notch filter 165 may have a notch width of approximately 808 nanometers (nm). The notch filter may be positioned between the long pass filter 163 and the image sensor 170.

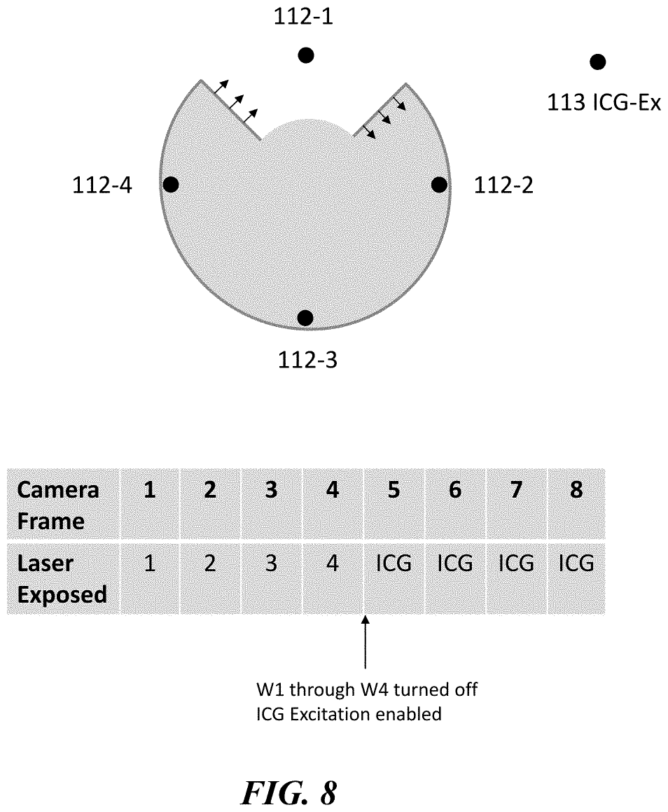

FIG. 8 illustrates a synchronization of (i) the exposure of one or more laser light sources 112-1, 112-2, 112-3, and 112-4 through the movable plate 120 with (ii) one or more camera frames captured by the image sensor or the camera. The plurality of illumination sources may include an indocyanine green (ICG) excitation light source 113 that is not optically aligned with the movable plate. The movable plate 120 may comprise a single notch. The imaging device may be configured to capture a first set of camera frames based on the exposure of the one or more laser light sources. Afterwards, the one or more laser light sources may be turned off, and the ICG excitation light source may be turned on. The imaging device may be configured to capture a second set of camera frames based on one or more ICG excitation light beams generated by the ICG excitation light source. The first set of camera frames and the second set of camera frames may be acquired at 120 frames per second (FPS). ICG emission characteristics (e.g., fluorescence caused by the interaction of one or more dyes with the ICG excitation light beam) may be imaged in any camera frames where the ICG excitation light source is turned on and/or enabled.

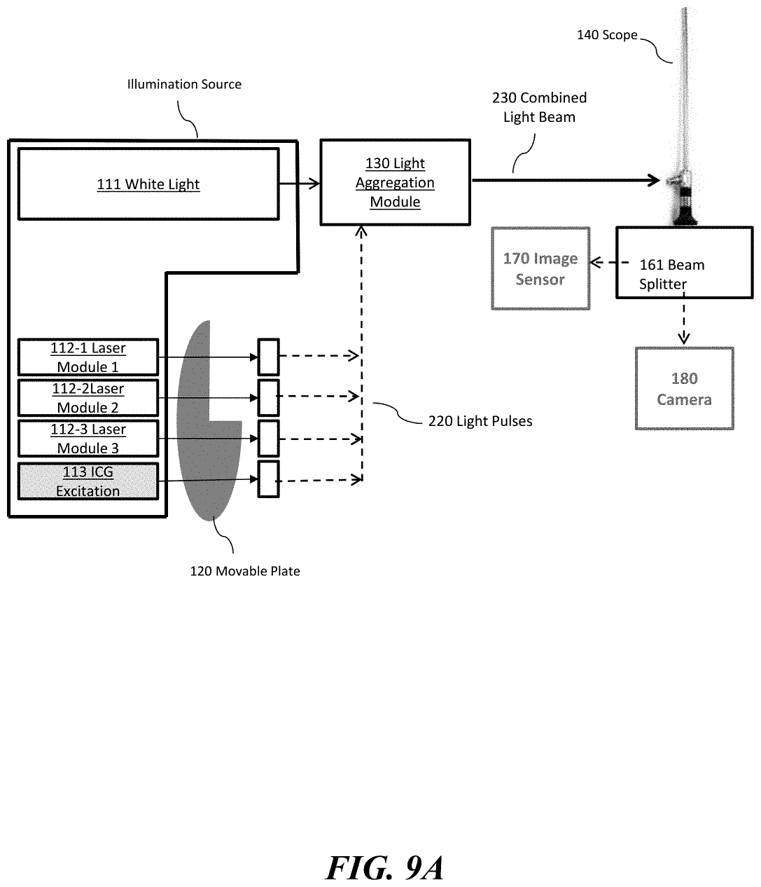

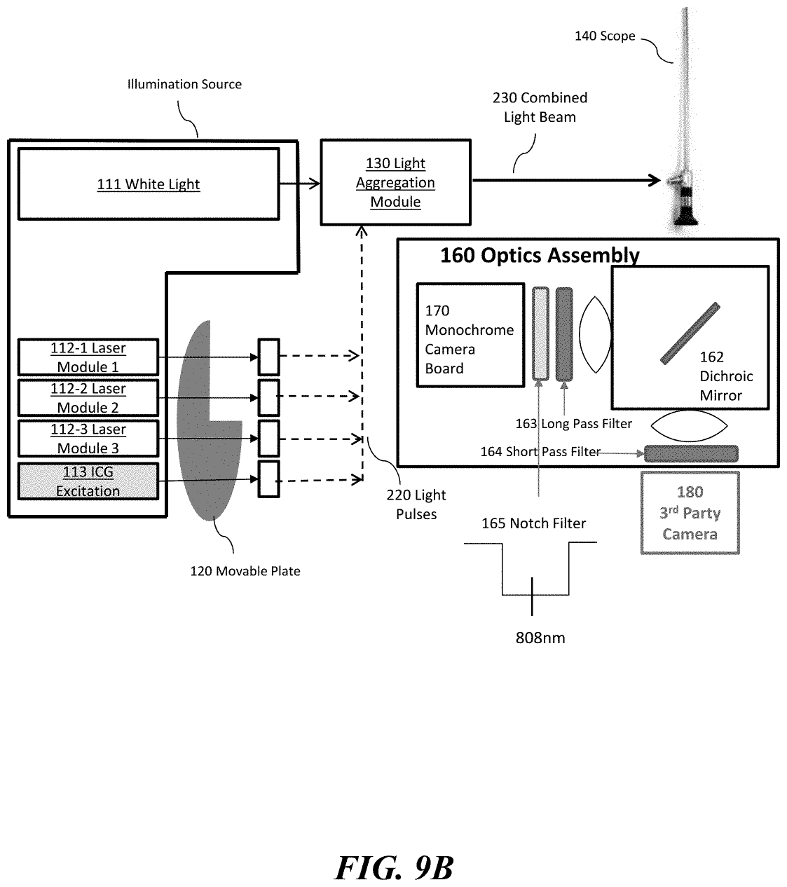

As illustrated in FIGS. 9A-9B, in some cases, the movable plate 120 may be optically aligned with (i) the one or more laser light sources 112-1, 112-2, and 112-3 and (ii) the ICG excitation light source 113. The plurality of illumination sources may comprise the white light source 111, the one or more laser light sources 112-1, 112-2, and 112-3, and the ICG excitation light source 113. In such cases, the movable plate and the white light source may not share a common optical axis. The white light beam from the white light source may be transmitted continuously without being affected or separated into pulses by the movable plate. The one or more light pulses 220 generated by the movable plate 120 may be obtained from (i) the one or more laser light beams and (ii) the ICG excitation light beam.

In such cases, the light aggregation module 130 may be configured to (i) combine (a) the one or more light pulses 220 obtained from the one or more laser light beams and the ICG excitation light beam with (b) the white light beam to generate (c) a combined light beam 230, and (ii) provide the combined light beam 230 to a scope 140. The scope may be insertable into the subject's body and configured to direct the combined light beam onto the target region.

As described above, a reflected light signal may be generated when the combined light signal 230 is emitted onto and reflected from the target region. The reflected light signal may comprise a first portion of the reflected light signal and a second portion of the reflected light signal. The first portion of the reflected light signal may comprise deflected light (e.g., backscattered light) that is generated when the target site is illuminated with one or more light pulses 220 obtained from one or more light beams generated by the laser light sources 112-1, 112-2, 112-3 and/or the ICG excitation light source 113. The second portion of the reflected light signal may comprise reflected light that is generated when the target site is illuminated with a different light (e.g., the white light beam). The scope 140 may be configured to direct the reflected light signal towards a beam splitter 161. The beam splitter may be configured to separate the reflected light signal into the first portion of the reflected light signal and the second portion of the reflected light signal. The first portion may be provided to an image sensor 170 to generate a first image. The second portion may be provided to a camera 180 to generate a second image. The image sensor 170 may be configured to provide the first image to an image processing module. The camera 180 may be configured to provide the second image to the image processing module. The image processing module may be configured to generate a combined image based on the first image and the second image.

FIG. 9B illustrates an optics assembly 160 that may be configured to receive the reflected light signal. The optics assembly 160 may comprise a dichroic mirror 162. In some cases, the dichroic mirror may be interchanged with a beam splitter, a half mirror, a dichroic beam splitter, or a multi-band beam splitter. The dichroic mirror 162 may be configured to receive the reflected light signal from the target site and (i) reflect the first portion of the light signals that is in a first electromagnetic spectral range toward an image sensor 170, and (ii) permit the second portion of the light signals in a second electromagnetic spectral range to pass through toward a camera 180. The camera 180 may or may not be integrated with the optics assembly 160. The optics assembly 160 may comprise a long pass filter 163. The long pass filter 163 may be positioned adjacent to and/or in front of the image sensor 170. The image sensor may be a monochrome camera board. The optics assembly 160 may comprise a short pass filter 164. The short pass filter 164 may be positioned adjacent to and/or in front of the camera 180. The optics assembly may comprise a notch filter 165. The notch filter 165 may have a notch width of approximately 808 nanometers (nm). The notch filter may be positioned between the long pass filter 163 and the image sensor 170.

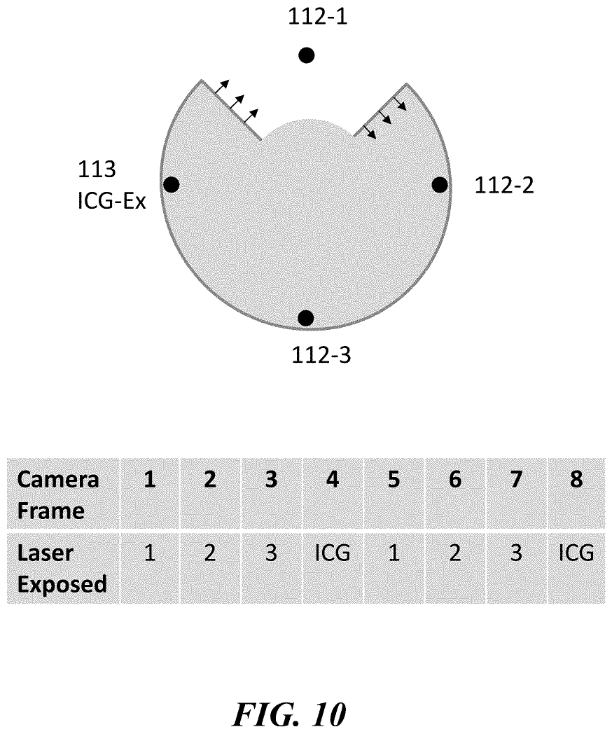

FIG. 10 illustrates a synchronization of (i) the exposure of (a) one or more laser light sources 112-1, 112-2, 112-3 and (b) an ICG excitation light source 113 with (ii) the acquisition of one or more camera frames captured by the image sensor or the camera. The one or more laser light sources and the ICG excitation light source may be optically aligned with the movable plate. The movable plate 120 may comprise a single notch. The imaging device may be configured to capture a first set of camera frames based on the exposure of the one or more laser light sources. The imaging device may be configured to capture a second set of camera frames based on the controlled exposure of the ICG excitation light source. The first set of camera frames may be acquired at 120 frames per second (FPS). The second set of camera frames may be acquired at 30 frames per second (FPS). The second set of camera frames may capture one or more ICG emission characteristics (e.g., fluorescence caused by the interaction of one or more dyes with the ICG excitation light beam). The second set of camera frames may be imaged in 1/N frames, wherein N may correspond to the number of laser light sources optically aligned with the movable plate 120.

In some cases, the movable plate may be optically aligned with (i) the one or more laser light sources and (ii) the white light source. The plurality of illumination sources may comprise the white light source, the one or more laser light sources, and the ICG excitation light source. In such cases, the movable plate and the ICG excitation light source may not share a common optical axis. The ICG excitation light beam from the ICG excitation light source may be transmitted continuously without being affected or separated into pulses by the movable plate. The one or more light pulses generated by the movable plate may be obtained from (i) the one or more laser light beams and (ii) the white light beam.

In such cases, the light aggregation module may be configured to (i) combine (a) the one or more light pulses obtained from the one or more laser light beams and the white light beam with (b) the ICG excitation light beam to generate (c) a combined light beam, and (ii) provide the combined light beam to a scope. The scope may be insertable into the subject's body and configured to direct the combined light beam onto the target region.

In some cases, the movable plate may be optically aligned with (i) the one or more laser light sources, (ii) the white light source, and (iii) the ICG excitation light source. In such cases, the one or more light pulses generated by the movable plate may be obtained from (i) the one or more laser light beams, (ii) the white light beam, and (iii) the ICG excitation light beam. Further, the light aggregation module may be configured to (i) combine (a) the one or more light pulses obtained from the one or more laser light beams with (b) the one or more light pulses obtained from the white light beam and the ICG excitation light beam to generate (c) a combined light beam, and (ii) provide the combined light beam to a scope. The scope may be insertable into the subject's body and configured to direct the combined light beam onto the target region.

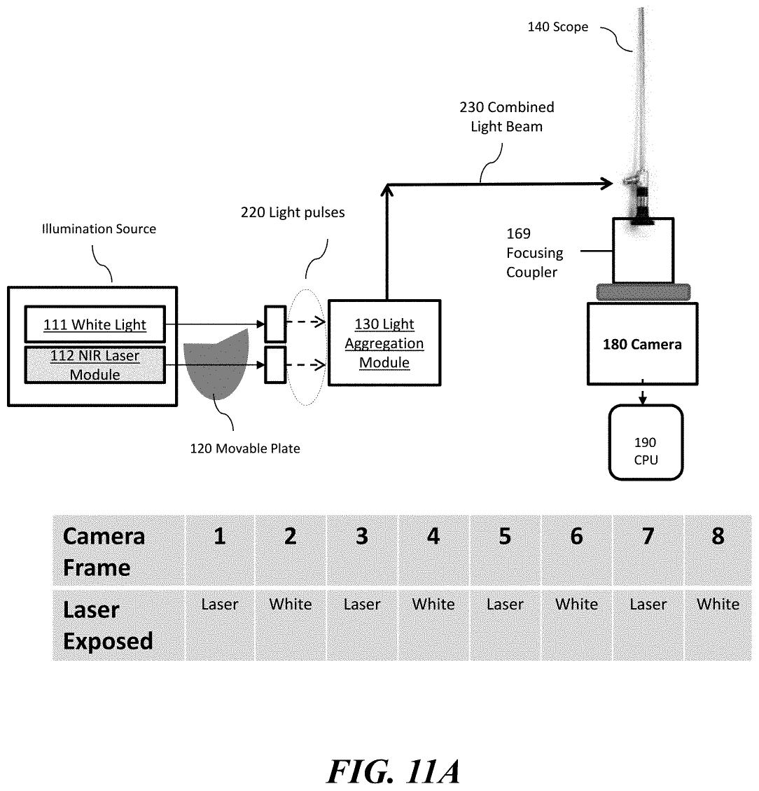

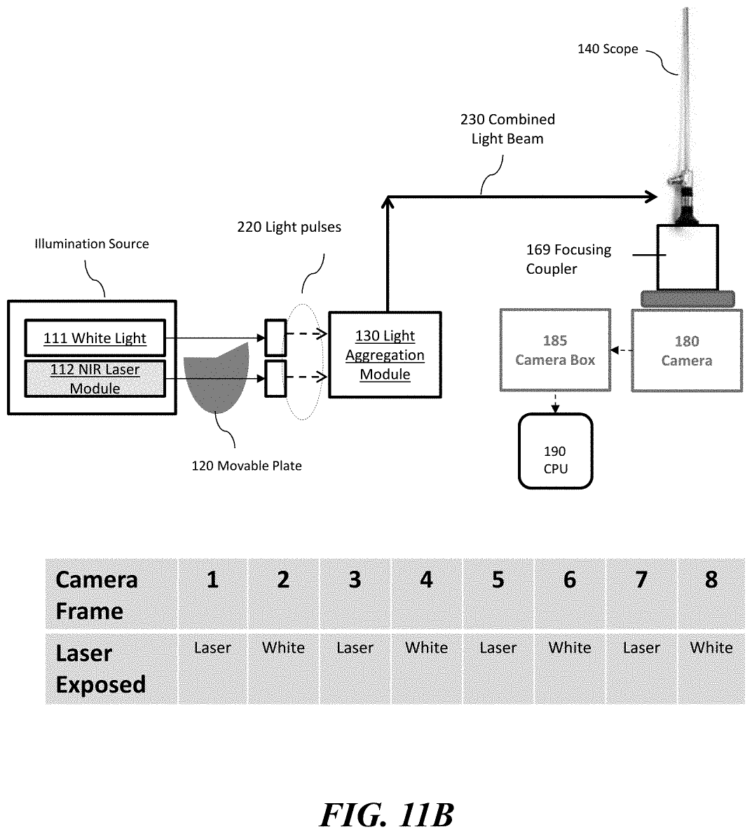

FIGS. 11A and 11B illustrate a white light source 111 and a laser light source 112 optically aligned with a movable plate 120. The white light source 111 and a laser light source 112 may be operated simultaneously and continuously. The white light source may be used to recover a standard color image and/or video of the target region. The laser light source may be used for laser speckle contrast imaging of the target region. The movable plate 120 may be configured to generate one or more light pulses 220 by controlling the exposure of the white light source 111 and the laser light source 112. The light aggregation module 130 may be configured to combine one or more sets of light pulses associated with the white light source 111 with one or more sets of light pulses associated with the laser light source 112 to generate a combined light beam 230. The combined light beam 230 may be provided to a scope 140, which scope may be configured to direct the combined light beam 230 to a target region in a subject's body.

A reflected light signal may be generated when the combined light signal 230 is emitted onto and reflected from the target region. The reflected light signal may comprise a first portion of the reflected light signal and a second portion of the reflected light signal. The first portion of the reflected light signal may comprise deflected light (e.g., backscattered light) that is generated when the target site is illuminated with one or more light pulses 220 obtained from one or more laser light beams generated by the laser light source 112. The second portion of the reflected light signal may comprise reflected light that is generated when the target site is illuminated with one or more light pulses 220 obtained from one or more white light beams generated by the white light source 111. The scope 140 may be configured to direct the reflected light signal towards an optics assembly. The optics assembly may comprise a focusing coupler 169. The focusing coupler may be configured to focus, modulate, and/or direct the first portion of the reflected light signal and/or the second portion of the reflected light signal to a camera 180. The camera may be configured to generate a combined image of the target region based on the first portion of the reflected light signal and the second portion of the reflected light signal. In some cases, the camera may be configured to provide the combined image, the first portion of the reflected light signal, and/or the second portion of the reflected light signal to a camera box 185. The camera box 185 may be configured to pre-process and/or modify the combined image, the first portion of the reflected light signal, and/or the second portion of the reflected light signal. The camera box 185 may be configured to provide the pre-processed image, the first portion of the reflected light signal, and/or the second portion of the reflected light signal to a central processing unit (CPU) 190. The CPU 190 may be configured to generate a modified and/or an overlaid (i.e., superimposed) image of the target region based on the first portion of the reflected light signal and/or the second portion of the reflected light signal.

As illustrated in FIGS. 11A and 11B, the camera 180 may be configured to capture a first set of frames associated with the white light source 111, and a second set of frames associated with the laser light source 112. The frames captured by the camera 180 may alternate between a frame from the first set of frames and a frame from the second set of frames. The exposure of the white light source 111 may be synchronized with the acquisition of one or more even-numbered frames. The exposure of the laser light source 112 may be synchronized with the acquisition of one or more odd-numbered frames. The camera 180 may be configured to capture frames at 120 frames per second. The camera 180 may be configured to capture 60 frames per second for the white light source 111 and another 60 frames per second for the laser light source 112.

In some cases, the camera 180 and/or the camera box 185 may be configured to calibrate for phase delay between the generation of one or more light pulses and the acquisition of one or more frames. In such cases, the camera 180 and/or the camera box 185 may be configured to (i) turn off one or more illumination sources and (ii) tunes, sweep, and/or optimize one or more delay parameters for the movable plate, until even-numbered frames are completely dark and odd-numbered frames are bright.

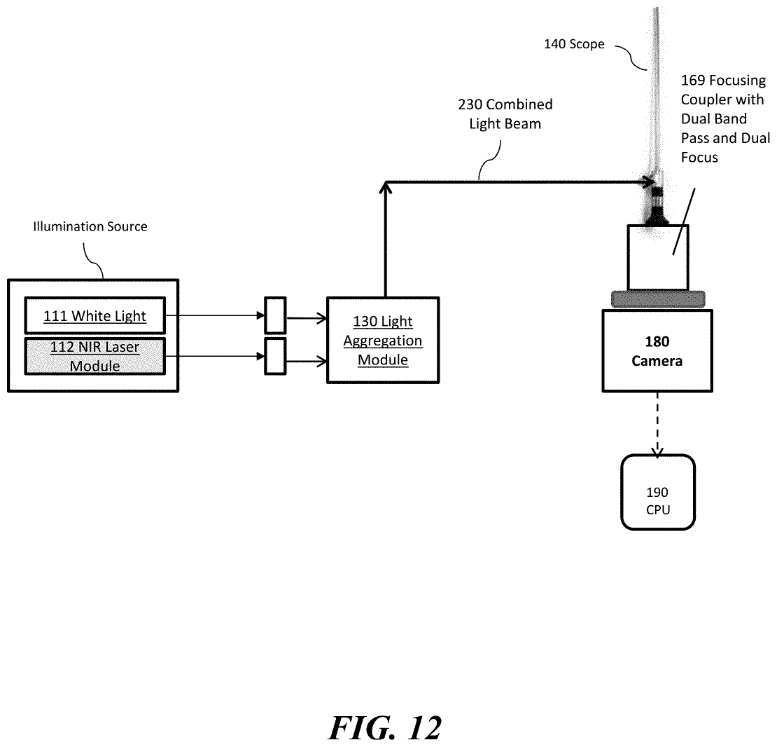

FIG. 12 illustrates a light aggregation module 130 configured to (a) aggregate a white light beam generated by a white light source 111 and a laser light beam generated by a laser light source 112 to (b) generate a combined light beam 230. The combined light beam 230 may be provided to a scope 140, which scope may be configured to direct the combined light beam 230 to a target region in a subject's body. The laser light beam and the white light beam may be transmitted as continuous beams with stable output power.

A reflected light signal may be generated when the combined light signal 230 is emitted onto and reflected from the target region. The reflected light signal may comprise a first portion of the reflected light signal and a second portion of the reflected light signal. The first portion of the reflected light signal may comprise deflected light (e.g., backscattered light) that is generated when the target site is illuminated with one or more laser light beams generated by the laser light source 112. The second portion of the reflected light signal may comprise reflected light that is generated when the target site is illuminated with one or more white light beams generated by the white light source 111. The scope 140 may be configured to direct the reflected light signal towards an optics assembly. The optics assembly may comprise a focusing coupler 169. The focusing coupler 169 may be configured to focus, modulate, and/or direct the first portion of the reflected light signal and/or the second portion of the reflected light signal to a camera 180. The focusing coupler 169 may comprise dual band pass filters and dual focusing elements. The camera 180 may be configured to generate a combined image of the target region based on the first portion of the reflected light signal and the second portion of the reflected light signal. In some cases, the camera may be configured to provide the combined image, the first portion of the reflected light signal, and/or the second portion of the reflected light signal to a CPU 190. The CPU 190 may be configured to process and/or modify the combined image, the first portion of the reflected light signal, and/or the second portion of the reflected light signal. The CPU 190 may be configured to generate a modified and/or an overlaid (i.e., superimposed) image of the target region based on the first portion of the reflected light signal and the second portion of the reflected light signal.

In another aspect, the present disclosure provides a system for illuminating a target region of a subject's body. The system may comprise a plurality of illumination sources comprising at least two of (i) a white light source configured to generate a white light beam and (ii) one or more light emitting diodes (LEDs) or laser light sources configured to generate one or more laser light beams; and a movable plate comprising one or more cut-outs. The movable plate may be optically aligned with one or more of the plurality of illumination sources and configured to (i) move relative to the one or more illumination sources and (ii) control a pulsing of the one or more illumination sources in synchronization with a pre-determined frame capture rate. In some cases, the movable plate may be configured to control the pulsing of the one or more illumination sources by adjusting one or more time intervals during which each of the plurality of illumination sources is optically aligned with the one or more cut-outs of the movable plate.

In another aspect, the present disclosure provides methods for illuminating a target region in a subject's body. The method may comprise: providing a plurality of illumination sources comprising (i) a white light source configured to generate a white light beam and (ii) one or more laser light sources configured to generate one or more laser light beams; directing one or more light beams generated by the plurality of illumination sources towards a movable plate comprising one or more cut-outs, wherein the movable plate is (i) optically aligned with one or more of the plurality of illumination sources, and (ii) configured to move so as to (a) control an exposure of the one or more illumination sources through the one or more cut-outs, relative to a pre-determined frame capture rate, and (b) generate one or more light pulses based on the controlled exposure of the one or more illumination sources; and providing the one or more light pulses to a light aggregation module, wherein the light aggregation module is configured to (i) combine the one or more light pulses obtained from each of the one or more light beams generated by the plurality of illumination sources to generate a combined light beam, and (ii) provide the combined light beam to a scope, wherein the scope is insertable into the subject's body and configured to direct the combined light beam onto the target region. In some cases, the plurality of illumination sources may further comprise an indocyanine green (ICG) excitation light source configured to generate an ICG excitation light beam.

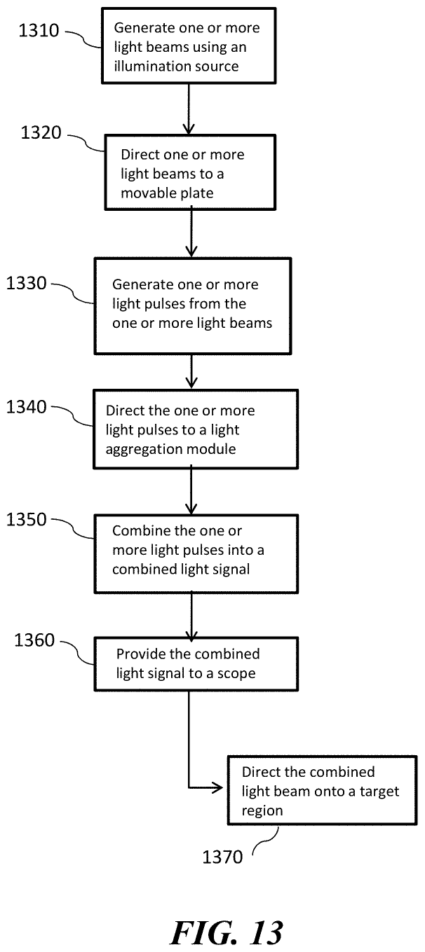

FIG. 13 illustrates an example of a method for illuminating a target region in a subject's body. The method may comprise: (a) generating one or more light beams using an illumination source (1310), (b) directing one or more light beams to a movable plate (1320), (c) generating one or more light pulses from the one or more light beams (1330), (d) directing the one or more light pulses to a light aggregation module (1340), (e) combining the one or more light pulses into a combined light signal (1350), (f) providing the combined light signal to a scope (1360), and (g) directing the combined light beam onto a target region (1370).

Computer Systems

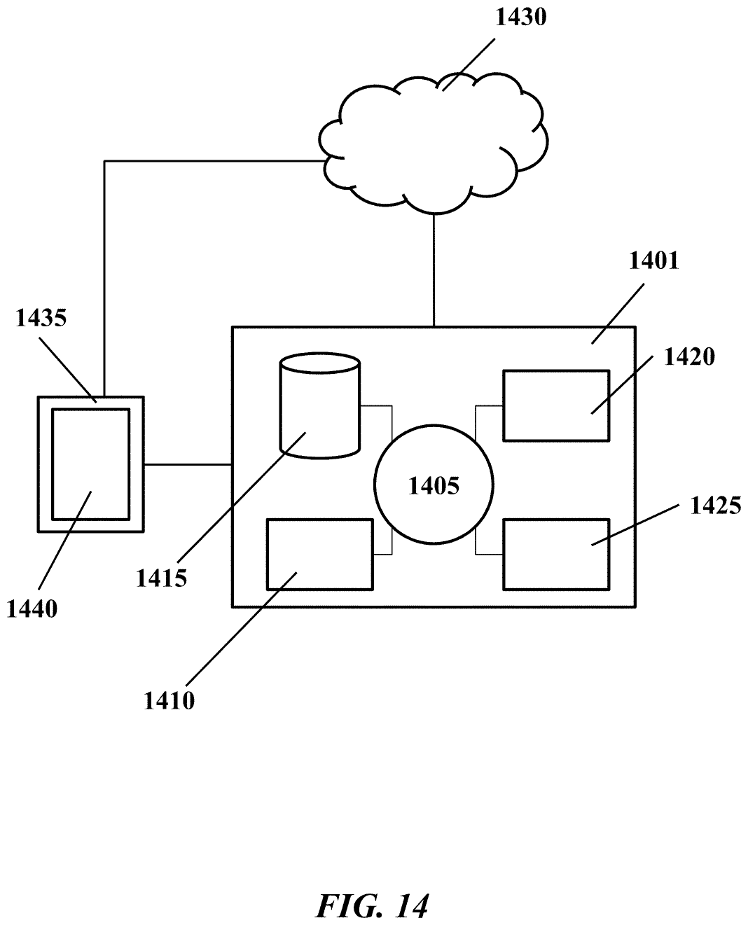

Another aspect of the present disclosure provides computer systems that are programmed or otherwise configured to implement methods of the disclosure. FIG. 14 shows a computer system 1401 that is programmed or otherwise configured to implement a method for medical imaging. The computer system 1401 may be configured to (a) generate one or more light beams using an illumination source, (b) direct one or more light beams to a movable plate, (c) generate one or more light pulses from the one or more light beams, (d) direct the one or more light pulses to a light aggregation module, (e) combine the one or more light pulses into a combined light signal, (f) provide the combined light signal to a scope, and (g) direct the combined light beam onto a target region in the subject's body. The computer system 1401 can be an electronic device of a user or a computer system that is remotely located with respect to the electronic device. The electronic device can be a mobile electronic device.

The computer system 1401 may include a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC).

The storage unit 1415 can store files, such as drivers, libraries and saved programs. The storage unit 1415 can store user data, e.g., user preferences and user programs. The computer system 1401 in some cases can include one or more additional data storage units that are located external to the computer system 1401 (e.g., on a remote server that is in communication with the computer system 1401 through an intranet or the Internet).

The computer system 1401 can communicate with one or more remote computer systems through the network 1430. For instance, the computer system 1401 can communicate with a remote computer system of a user (e.g., a patient, a subject, a doctor, a medical operator, a surgical operator, a nurse, a surgeon, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1401 via the network 1430.

Methods as described herein can be implemented by way of machine (e.g., computer processor) executable code stored on an electronic storage location of the computer system 1401, such as, for example, on the memory 1410 or electronic storage unit 1415. The machine executable or machine readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1401 can include or be in communication with an electronic display 1435 that comprises a user interface (UI) 1440 for providing, for example, a portal for modulating and controlling the plurality of illumination sources and/or a movement of the movable plate relative to the plurality of illumination sources. In some cases, the portal may be used to render, view, monitor, and/or manipulate one or more images or camera frames generated in part based on a reflection and/or a deflection of the combined light beam from a target region inside the subject's body. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1405. The algorithm may be configured to (a) generate one or more light beams using an illumination source, (b) direct one or more light beams to a movable plate, (c) generate one or more light pulses from the one or more light beams, (d) direct the one or more light pulses to a light aggregation module, (e) combine the one or more light pulses into a combined light signal, (0 provide the combined light signal to a scope, and (g) direct the combined light beam onto a target region in the subject's body.

While preferred embodiments of the present disclosure have been shown and described herein, it will be obvious to those skilled in the art that such embodiments are provided by way of example only. It is not intended that the present disclosure be limited by the specific examples provided within the specification. While the present disclosure has been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein may be employed in practicing one or more aspects of the present disclosure. It is therefore contemplated that the present disclosure shall also cover any such alternatives, modifications, variations or equivalents. It is intended that the following claims define the scope of the present disclosure and that the methods and structures within the scope of these claims and their equivalents be covered thereby.

What is claimed is:

1. A system for illuminating a target region of a subject's body, comprising:
a plurality of illumination sources comprising at least two of (i) a white light source configured to generate a white light beam and (ii) one or more light emitting diodes (LEDs) or laser light sources configured to generate one or more laser light beams; and
a movable plate comprising one or more cut-outs, wherein the movable plate is (i) optically aligned with one or more of the plurality of illumination sources and (ii) configured to move so as to (a) control an exposure of the one or more illumination sources through the one or more cut-outs, relative to a pre-determined frame capture rate, and (b) generate one or more light pulses based on the controlled exposure of the one or more illumination sources.

2. The system of claim 1, wherein the movable plate is configured to rotate relative to the one or more illumination sources.

3. The system of claim 1, wherein the one or more cut-outs comprise a notch on the movable plate.

4. The system of claim 1, wherein the one or more cut-outs comprise a plurality of notches arranged on different portions of the movable plate.

5. The system of claim 1, wherein the one or more cut-outs comprise one or more annular-shaped openings on the movable plate.

6. The system of claim 1, wherein the movable plate comprises a low transmittance material that is configured to prevent transmission of light through one or more solid portions of the movable plate.

7. The system of claim 1, wherein the one or more laser light sources comprise two or more laser light sources that are configured to generate two or more laser light beams having different wavelengths.

8. The system of claim 7, wherein the two or more laser light sources comprise a gas laser, a chemical laser, a liquid laser, a dye laser, a metal-vapor laser, a solid-state laser, or a semiconductor laser.

9. The system of claim 7, wherein the two or more laser light sources comprise an infrared laser, a near-infrared laser, a short-wavelength infrared laser, a mid-wavelength infrared laser, a long-wavelength infrared laser, or a far-infrared laser.

10. The system of claim 7, wherein the two or more laser light sources are configured to generate two or more laser light beams with a wavelength between about 700 nanometers (nm) and about 1 millimeter (mm).

11. The system of claim 1, wherein the movable plate is optically aligned with the one or more laser light sources.

12. The system of claim 11, wherein the movable plate and the white light source do not share a common optical axis.

13. The system of claim 11, wherein the white light source is disposed relative to the movable plate such that the white light beam does not pass through the movable plate.

14. The system of claim 11, wherein the white light beam from the white light source is transmitted continuously without being affected or separated into pulses by the movable plate.

15. The system of claim 11, wherein the one or more light pulses are obtained from the one or more laser light beams.

16. The system of claim 15, further comprising:
a light aggregation module configured to (i) combine (a) the one or more light pulses obtained from the one or more laser light beams with (b) the white light beam to generate (c) a combined light beam, and (ii) provide the combined light beam to a scope, wherein the scope is insertable into the subject's body and configured to direct the combined light beam onto the target region.

17. The system of claim 1, wherein the movable plate is optically aligned with (i) the white light source and (ii) the one or more laser light sources.

18. The system of claim 17, wherein the one or more light pulses are obtained from (i) the white light beam and (ii) the one or more laser light beams.

19. The system of claim 18, further comprising:
a light aggregation module configured to (i) combine (a) the one or more light pulses obtained from the white light beam with (b) the one or more light pulses obtained from the one or more laser light beams, to generate (c) a combined light beam, and (ii) provide the combined light beam to a scope, wherein the scope is insertable into the subject's body and configured to direct the combined light beam onto the target region.

20. The system of claim 1, wherein the plurality of illumination sources further comprises (iii) an indocyanine green (ICG) excitation light source configured to generate an ICG excitation light beam.
